(19) United States
(12) Patent Application Publication
(10) Pub. No.: US 2025/0237642 A1
(43) Pub. Date: Jul. 24, 2025

(54) PREDICTIVE MODEL FOR VARIANTS ASSOCIATED WITH DRUG RESISTANCE AND THERANOSTIC APPLICATIONS THEREOF

(71) Applicant: Emory University, Atlanta, GA (US)

(72) Inventors: Raymond F. Schinazi, Miami, FL (US); Dharmeshkumar Jethalal PATEL, Atlanta, GA (US)

(21) Appl. No.: 18/844,863

(22) PCT Filed: Mar. 8, 2023
(86) PCT No.: PCT/US2023/014828

§ 371 Date: Sep. 6, 2024

Related U.S. Application Data
(60) Provisional application No. 63/317,739, filed on Mar. 8, 2022.

(51) Int. Cl. G01N 33/50; C12N 15/10

(52) U.S. Cl. CPC G01N 33/5091; C12N 15/1058; C12N 15/1089

(57) ABSTRACT

Methods for predicting mutations in viruses, such as Coronaviruses, upon exposure to antiviral drugs, are disclosed. Mutated, non-naturally occurring viruses including those mutations, and methods of treatment with drugs that remain effective against the mutated viruses, are disclosed. These predictive methods can be useful in properly treating Covid patients with small molecule antiviral compounds that are effective against the particular SARS-COV-2 variant infecting the patient.

### FIELD OF THE INVENTION

Methods for predicting mutations in viruses, such as Coronaviruses, upon exposure to antiviral drugs, mutated, non-naturally occurring viruses including those variants, and methods of treatment with drugs that remain effective against the mutated viruses, are disclosed.

### BACKGROUND

The Severe Acute Respiratory Syndrome Coronavirus-2 (SARS-COV-2) pandemic has caused over 187 million confirmed cases of COVID-19, and more than 4 million deaths. The mortality and morbidity of this virus is due in part to the interaction of the spike protein with the ACE-2 receptor, in part due to blood clotting, and in part due to the cytokine storm resulting from the body's natural defenses against the virus.

There have been hundreds of millions of doses of RNA-based vaccines administered throughout the world. These viruses have focused on the transient production of the spike protein sequence from the original virus, as this is a relatively abundant target on the surface of the virus. The production, in vivo, of spike protein leads to the production of antibodies to this spike protein. While these vaccines appear to have been relatively successful against the original strain, with the original spike protein sequence, the viruses have mutated the protein sequence of the spike protein, and developed resistance against the antibodies produced by the original vaccines. While the vaccines were reasonably effective against many of these variants, including the Delta variant, the original two dose regimen of the Pfizer vaccine is not very effective against the Omicron variant. Even patients who have received the booster shot can still be infected, and transmit the disease, but are likely to have somewhat lesser symptoms, and lower rates of hospitalization and death, relative to individuals who are a) unvaccinated and b) have not had a prior infection.

Because the mutated viruses can infect both vaccinated and unvaccinated individuals, there is an interest in early treatment options, including antiviral agents that are effective against the mutated viruses. These agents do not typically bind the spike protein. Rather, they bind to one or more enzymes, such as the protease and/or polymerase enzymes, required for the virus to propagate. However, while these antiviral agents may be active against viruses with mutated spike proteins, it is predicted that their administration will result in mutations in the virus' protease and/or polymerase enzymes, as well as other enzymes critical for virus survival and their fitness.

Since multiple therapeutic approaches will likely be implemented to address SARS-COV-2 and future zoonotic outbreaks, it can be useful to know what mutations might be formed upon exposure to a given antiviral agent. It can also be useful to have diagnostic methods to identify patients with these variants, and therapeutic approaches for treating patients with an antiviral agent to which the mutated virus is still susceptible. The present disclosure provides such diagnostic and therapeutic methods.

### SUMMARY

In one embodiment, methods for predicting mutations that would likely occur in a coronavirus, picornavirus, or for example polymerase, protease and helicase enzymes, upon exposure to a specific inhibitor, are disclosed. For the case of a protease inhibitor, methods involve obtaining a crystal structure of the protease enzyme with the protease inhibitor locked into the binding pocket, and obtaining a crystal structure of the protease with one or more of the substrates to which the protease has activity, i.e., where the protease is known to cleave a protein.

Using molecular modeling software, putative amino acid mutations in various amino acids present in the binding pocket are made. Where the change in the protease structure alters the binding pocket in such a way that the protease inhibitor has significantly less binding affinity, for example, a free energy change (i.e., ΔΔG)>0, this corresponds to a variant that will emerge upon exposure to the protease inhibitor. However, the virus is not likely to create a variant that will stop the protease from functioning, so the next step in the process is to evaluate crystal structures of the protease and the substrate to which the protease has activity, looking at the protease activity of the wild-type protease and the protease with the mutation(s) identified in the initial step. If the mutation(s) result in a protease with less binding affinity to the substrate is ΔΔG≤0, then the affinity for the substrates was either maintained or increased. Where a protease has multiple substrates to which it binds, not all of the substrates need to be affected for the mutation to be an unlikely mutation for the virus to make. For example, for the Coronavirus 3CLpro protease, which cleaves eleven different substrates, this predictive model assumes that a mutation in the protease cannot result in a ΔΔG≤0 for more than 3 of these substrates. If the binding of the 3CLpro to more than three of the substrates are so affected, then the mutation is not likely to occur in response to exposure to the protease inhibitor.

Once a list of mutations is identified, where the mutations cause the protease inhibitor to lose its binding affinity to the protease, but the protease still maintains activity against the substrate(s), these are potential mutations to look for in patients infected with the virus and treated with the putative antiviral agent. That is, if a patient has a viral infection, and the virus has a mutation associated with poor binding of the protease inhibitor, then other treatment options should be considered.

Where a plurality of protease inhibitors are commercially available, and the crystal structure of the protease and the inhibitors is also available, the plurality of protease inhibitors can be subjected to this process, and a library of mutations associated with resistance to each protease can be prepared.

When a patient has been identified as being infected, a PCR test can be performed to identify whether there are variants present in the coronavirus infecting the patient that indicate the virus will not be susceptible to one or more protease inhibitors. The library of variants can be screened, and this information used to identify one or more protease inhibitors to which the particular coronavirus has not developed resistance (i.e., there is no cross-resistance). The patient can then be treated using one or more of these protease inhibitors or another class of antiviral agent (e.g., a polymerase inhibitor).

In performing a PCR test, it can be useful to have primers to which the viral DNA will bind if it has a given mutation. Primers bind complementarily to the viral DNA, and typically have from 3-20 bases to the left and to the right of the mutation. One can also sequence the virus and compare it to the sequence of the parent CoV-2 Wuhan strain. Once a list of potential variants has been identified, appropriate primers can be designed. These primers can be labeled, for example, with a fluorescent label. In one embodiment, the disclosure relates to primers corresponding to one or more of the predicted mutations, and in another embodiment, the disclosure relates to a PCT test involving screening a biological sample to identify the presence of one or more viral variants.

In yet another embodiment, isolated coronaviruses including one or more of the predicted variants are disclosed. These viral variants can be used, for example, as reference controls in laboratories to confirm that the PCR test being performed is capable of detecting the viral variants.

Because there is a high degree of homology between proteases for known coronaviruses, as well as picornaviruses and caliciviruses (e.g., entero and noroviruses), the assay can be used not only for SARS-COV-2, but for other coronaviruses and picornaviruses or caliciviruses.

In other embodiments, the methods can be used to detect mutations in enzymes, such as protease and reverse transcriptase, found in retroviruses such as HIV and HBV, as well as proteases found in HCV.

We can also confirm the impact of the mutations found to be selected by the protease inhibitor by expressing wild type and mutant proteases and evaluating their phenotype using enzymic methodologies (see Loschwitz et al., “Novel inhibitors of the main protease enzyme of SARS-COV-2 identified via molecular dynamics simulation-guided in vitro assay,” Bioorg Chem. 2021 June).

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a schematic illustration of a crystal structure of 3CLpro with protease inhibitor PF07321332.

FIG. 2 is a schematic illustration of a crystal structure of 3CLpro with substrate peptides nsp4-nsp5.

FIG. 3 is a schematic illustration of a crystal structure of 3CLpro with substrate peptides nsp6-nsp7.

FIG. 4 is a schematic illustration of a crystal structure of 3CLpro with substrate peptides nsp8-nsp9.

FIG. 5 is a schematic illustration of a crystal structure of 3CLpro with substrate peptides nsp9-nsp10.

FIG. 6 is a schematic illustration of a crystal structure of 3CLpro with substrate peptides nsp14-nsp15.

FIG. 7 is a schematic illustration of a crystal structure of 3CLpro with substrate peptides nsp15-nsp16.

FIG. 8 is a flow chart that illustrates an embodiment of the theranostic methods described herein, including routines for entering a user-defined therapeutic treatment regimen and for entering a “non-recommended” therapeutic treatment regimen.

FIG. 9 is a flow chart that illustrates an embodiment of a system or apparatus for use in the theranostic methods described herein.

FIG. 10 is a flow chart that illustrates an embodiment of the theranostic methods described herein, illustrating a client-server environment within which the system of FIG. 9 may operate, and wherein a central server is accessible by at least one local server via a computer network, such as the Internet, and wherein each local server is accessible by at least one client.

FIG. 11 is a chart that the T109I mutant is more susceptible to inhibition of HBeAg production by GLP-26, versus GLS4, when compared to the wild type.

FIG. 12 is a chart showing that the T109 mutant is susceptible to reduction of cccDNA by GLP-26, where drugs are used as a concentration of 10 μM.

FIG. 13 is a chart showing predicted resistance mutations in SARS-COV2 3CLpro for both nirmatrelvir and Compound 1.

FIG. 14 is a flow chart of computational approach for the prediction of drug resistance mutations.

FIG. 15A is a chart showing selected binding site residues in HIV-RT to predict resistance mutations for (-)-FTC, and FIG. 15B shows the chemical structures of (-)-FTC and natural substrate 2′-deoxycytidine (dC).

FIG. 16 is a chart showing predicted binding free energy change (ΔΔG) in kcal/mol of native substrate dCTP versus (-)-FTC-TP for single point mutations in HIV-RT. Violet circles represent known clinical (-)-FTC resistance mutations.

FIG. 17A is a chart showing selected binding site residues between two monomer proteins of the HBV core to predict GLP-26 resistance mutations. The monomers are represented in gray and yellow, and their respective residues are in green. FIG. 17B shows the chemical structure of GLP-26.

FIG. 18 is a chart showing the inhibition of HBeAg secretion in HBV wild-type and core protein mutants 9% inhibition+/−SD) at 10 μM GLP-26.

FIG. 19A is a chart showing the selected binding site residues in SARS-COV-2 3CLpro to predict nirmatrelvir resistance mutations. FIG. 19B shows the chemical structure of nirmatrelvir.

### DETAILED DESCRIPTION

In one embodiment, a predictive model for identifying mutations in viral enzymes, following exposure to one or more compounds that inhibit the enzymes, is disclosed. In this predictive model, the first step is to identify an enzyme for which there are crystal structures with the one or more enzyme inhibitors complexed with it. Molecular modeling is performed to modify the amino acids in the binding pocket such that the mutation(s) result in the enzyme inhibitor having significantly less binding affinity than prior to the mutation, for example, a free energy change (i.e., ΔΔG)>0, such that it is no longer as effective against the enzyme due to a poor binding affinity for the binding pocket. These are potential mutations a virus might use to avoid succumbing to a particular drug. However, as the mutations are only likely to occur if the enzyme does not lose a substantial amount of its activity, the predictive model also involves evaluating the same mutations in the enzyme when it is complexed with the substrate with which it exerts activity. For example, where the enzyme is a protease, and is known to cleave certain peptide sequences, the ability of the mutated enzyme to cleave the peptide sequences is also evaluated, using crystal structures of the enzyme and the substrate, and modifying the amino acid sequence in the same way as was done to predict mutations associated with drug resistance. Mutations that alter the binding affinity of the drug, but not the binding affinity of the enzyme to the substrate, are identified as potential mutations of interest.

In one aspect of this embodiment, a predictive model for identifying mutations in coronavirus proteases, such as 3CLpro, the protease used by SARS-COV-2, following exposure to one or more protease inhibitors, is disclosed. In this predictive model, the first step is to identify an enzyme for which there are crystal structures with the one or more protease inhibitors complexed with it. Molecular modeling is performed to modify the amino acids in the binding pocket such that the mutation(s) result in the protease inhibitor having significantly less binding affinity than prior to the mutation, for example, a free energy change (i.e., ΔΔG)>0, such that it is no longer as effective against the protease due to a poor binding affinity for the binding pocket.

Once mutations are found that cause the protease inhibitor to lose its binding affinity, or a significant amount thereof, the next step is to identify which mutations do not cause a significant decrease in the protease's ability to cleave proteins.

Looking at the protease found in SARS-COV-2, there are 11 peptides that are cleaved by 3Clpro. Ideally, one would have a crystal structure for all of these substrates complexed to the protease. Crystal structures are available for six of these substrates in complex with the protease, and the cleavage sites associated with each of the substrates is shown in the table below:
6 crystal structure of 3CLpro with substrate peptides

6 crystal structure of 3CLpro with substrate peptides

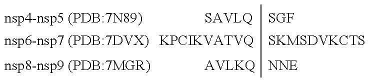

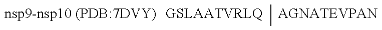

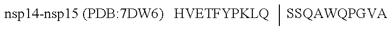

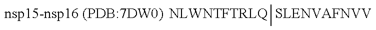

The dividing line indicates a cleavage line in the substrate peptides.

Using molecular modeling, Applicant has discovered that the protease 3CLpro was able to mutate to avoid the Pfizer protease inhibitor, PF-07321332, at the following positions on the protease amino acid sequence:

| | PREDICTED | | PREDICTED |
|---|---|---|---|
| | RESISTANT | | RESISTANT |
| WT | MUTATIONS | WT | MUTATIONS |
| S144 | Y, W | P52 | R, S |
| H163 | Y, Q | H172 | Q, N |
| L167 | Q, P, R, I, M, H | H164 | N |
| T190 | N, I, R, K, A | Q189 | H |
| R188 | H, K, N, L | A191 | G, D |
| M49 | N, R, K, F | H164 | L, D, Q, K, T, E |
| P168 | S, Q, R, A, H | D187 | G, Q, A |
| Q192 | H, K, Y, L, P | L141 | Q, H |
| E166 | G, A, K, V, N | M165 | L |
| F140 | S, I, V, L | F181 | L, I, V, S, C |
| G143 | A, C | N142 | I |
| Y54 | D, C, E, Q, N, F, G, | | |
| | H, M, S, W, K | | |

In this table, bold letters indicate mutations that inhibit binding of the protease inhibitor to a greater degree than the other listed mutations. WT stands for the wild-type protease. Underlined letters are the mutations exist in nature without drug treatment.

The predictive method, and methods for using this information to diagnose and treat patients, is discussed in more detail below.

### I. Protease Inhibitors for SARS-COV2

3CLPro is a prominent protease which cleaves polyproteins to generate mature nonstructural proteins involved in the replication and transcription of coronaviruses. It can catalytically cleave a peptide bond between a glutamine at position P1 and a small amino acid (serine, alanine, or glycine) at position P1′. Among other cleavage sites, it can self-cleave the peptides TSAVLQ-SGFRK-NH2 and SGVTFQ-GKFKK. The protease is important in the processing of the coronavirus replicase polyprotein (POC6U8). It is the main protease in coronaviruses and corresponds to nonstructural protein 5 (nsp5). It cleaves the coronavirus polyprotein at 11 conserved sites. The 3CL protease has a cysteine-histidine catalytic dyad at its active site. The sulfur of the cysteine acts as a nucleophile and the imidazole ring of the histidine as a general base.

The rigorous specificity for recognizing the P1-Gln substrate residue at the cleavage site endows the high conservation of the ligand binding site among known coronaviruses. For this reason, it is a therapeutic target for treating COVID-19 and other coronavirus-caused diseases.

Certain protease inhibitors have been reviewed (Xiong et al., “In silico screening-based discovery of novel covalent inhibitors of the SARS-COV-2 3CL protease, 2022 Jan. 23, Eur J Med Chem. 2022; 231:114130. doi: 10.1016/j.ejmech.2022.114130. Some 3CLpro inhibitors are peptidomimetic compounds (see, for example, Pillaiyar et al., “Recent discovery and development of inhibitors targeting coronaviruses,” Drug Discov. Today. 2020; 25:668-688, Liu et al., “The development of Coronavirus 3C-Like protease (3CL (pro)) inhibitors from 2010 to 2020,” Eur. J. Med. Chem. 2020; 206:112711, and Xiong et al., “What coronavirus 3C-like protease tells us: from structure, substrate selectivity, to inhibitor design,” Med. Res. Rev. 2021; 41:1965-1998). They are often designed by adding a covalent warhead to a substrate mimic. The known SARS-COV-2 3CLpro inhibitors like N3, 11a, 13b, PF-00835231, PF-07321332, and TGEV 3CLpro inhibitors, such as Cbz-VNSTLQ-CMK, contain a warhead of Michael acceptor, aldehyde, a-ketoamide, hydroxymethylketone, nitrile, and chloromethyl ketone, respectively. In contrast, non-peptidomimetic inhibitors have been rather derived from high-throughput screening/virtual screening of repurposing drugs/natural products/compound database. The covalent warhead endows the superiority in prolonged residence time. Several SARS-COV-2 3CLpro non-peptidomimetic covalent inhibitors like ebselen, PX-12, carmofur, myricetin, and ester derivatives thereof have been identified mostly by the high-throughput screening.

Representative protease inhibitors include GC376, rupintrivir, lufotrelvir, PF-07321332, AG7404, Nirmatrelvir, Carmofur, Ebselen, GC376, GRL-0617, Rupintrivir, and Theaflavin digallate.

Coronavirus protease inhibitors are also described, for example, in PCT WO 2004/093860 by Pfizer, PCT WO 2004/101742 by Cytovia, US 2006/0014821 by Agouron Pharmaceuticals, PCT WO 2005/041904 by FulcrumPharmaceuticals, PCT WO 2005/066123 3 by TaigenBiotechnology, PCT WO 2005/113580 by Pfizer, US 2006/0019967 by National Health Research Institutes, Taiwan, PCT WO 2006/042478 by Tsinghua University, Shanghai Institute of OrganicChemistry, CN 1965833A by PekingUniversity, PCT WO 2006/061714 by Pfizer, PCT WO 2006/095624 by Tokyo Medical and Dental University, PCT WO 2007/075145 by Singapore Polytechnic and Shanghai Institute of Materia Medica, CN 103159665B by Tianjin International Joint Academy of Biotechnology and Medicine, PCT WO 2013/049382 by Kansas State University, The Ohio State University, and Wichita State University, KR 1020130002975 by Chonnam National University, PCT WO 2013/166319 by Kansas State University and Wichita State University, CN 106176728A by Institute of Microbiology, Chinese Academy of Sciences, PCT WO 2017/114509 by Shanghai Institute of Materia Medica, University of Lübeck, US 2017/0313685by the Purdue Research Foundation, PCT WO 2017/222935 by Kansas State University and Wichita State University, CN 108785293A by Tianjin International Joint Academy of Biomedicine, PCT WO 2018/042343 by GSK, PCT WO 2020/030143 by Shanghai Institute of Materia Medica and FudanUniversity, PCT WO 2021/176369 by Pfizer, Chem Med Chem Review doi.org/10.1002/cmdc.202100576, and Chem Med Chem 2022, 17, e202100576, as described in Chia et al., “A Patent Review on SARS Coronavirus Main Protease (3CLpro) Inhibitors,” Chem Med Chem 2022,17, e2021005.

The predictive model described herein can be used to evaluate any protease inhibitor for potential mutations that would occur when patients are treated with the protease inhibitor, so long as crystal structures for the protease complexed with the protease inhibitor are available.

### IA. Reverse Transcriptase Inhibitors for HIV/HBV

The following reverse transcriptase inhibitors are being or have been used in HIV treatment:

Zidovudine, also called AZT, ZDV, and azidothymidine, has the trade name Retrovir. Zidovudine was the first antiretroviral drug approved by the FDA for the treatment of HIV.

Didanosine, also called ddI, with the trade names Videx and Videx EC, was the second FDA-approved antiretroviral drug. It is an analog of adenosine.

Zalcitabine, also called ddC and dideoxycytidine, has the trade name Hivid. This drug has been discontinued by the manufacturer.

Stavudine, also called d4T, has trade names Zerit and Zerit XR.

Lamivudine, also called 3TC, has the trade name Zeffix and Epivir. It is approved for the treatment of both HIV and hepatitis B.

Abacavir, also called ABC, has the trade name Ziagen, is an analog of guanosine.

Emtricitabine, also called FTC, has the trade name Emtriva (formerly Coviracil). Structurally similar to lamivudine, it is approved for the treatment of HIV and undergoing clinical trials for hepatitis B.

Entecavir, also called ETV, is a guanosine analog used for hepatitis B under the trade name Baraclude. It is not approved for HIV treatment.

Truvada, made of emtricitabine and tenofovir disoproxil fumarate, is used to treat and prevent HIV. It is approved for HIV prevention in the US and manufactured by Gilead.

Azvudine, also called RO-0622. It has been investigated as a possible treatment of AIDS, hepatitis C, and Sars-Cov-2.

The predictive model described herein can be used to evaluate any reverse transcriptase inhibitor for potential mutations that would occur when patients are treated with the reverse transcriptase inhibitor, so long as crystal structures for the reverse transcriptase complexed with the reverse transcriptase inhibitor are available.

### II. Combination Therapy for Particular Use in Treating Coronaviridae Infections

In addition, or in place of, protease inhibitors, patients can be treated with additional compounds known to be useful for treating coronaviridae infection. In some embodiments, the compounds discussed below can be used in combination therapy to treat Covid-19 infections, or other respiratory infections with similar pathology, particularly where mutations in the virus are associated with resistance to one or more protease inhibitors.

In one aspect of this embodiment, combination therapy with a protease inhibitor, or monotherapy if the virus shows resistance to one or more protease inhibitors, can include an active agent selected from the group consisting of fusion inhibitors, entry inhibitors, polymerase inhibitors, antiviral nucleosides, such as remdesivir, GS-441524, N4-hydroxycytidine, and other compounds disclosed in U.S. Pat. No. 9,809,616, and their prodrugs, viral entry inhibitors, viral maturation inhibitors, JAK inhibitors, angiotensin-converting enzyme 2 (ACE2) inhibitors, SARS-COV-specific human monoclonal antibodies, including CR3022, and agents of distinct or unknown mechanism.

Umifenovir (also known as Arbidol) is a representative fusion inhibitor.

Representative entry inhibitors include Camostat, luteolin, MDL28170, SSAA09E2, SSAA09E1 (which acts as a cathepsin L inhibitor), SSAA09E3, and tetra-O-galloyl-β-D-glucose (TGG). The chemical formulae of certain of these compounds are provided below:

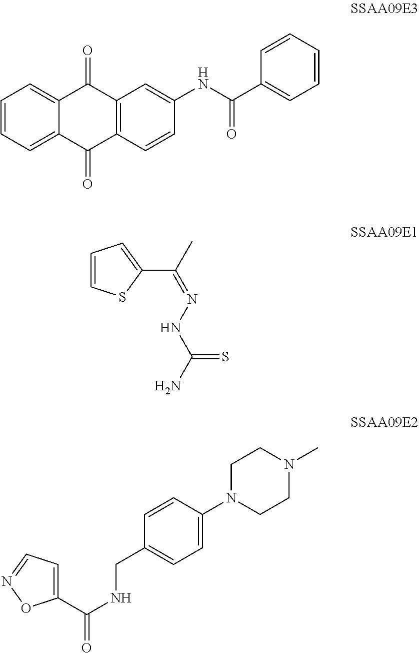 [SMILES: O=C(Nc1ccc2c(c1)C(=O)c1ccccc1C2=O)c1ccccc1 ; C/C(=N/NC(N)=S)c1cccs1 ; CN1CCN(c2ccc(CNC(=O)c3ccno3)cc2)CC1]

Other entry inhibitors include the following:

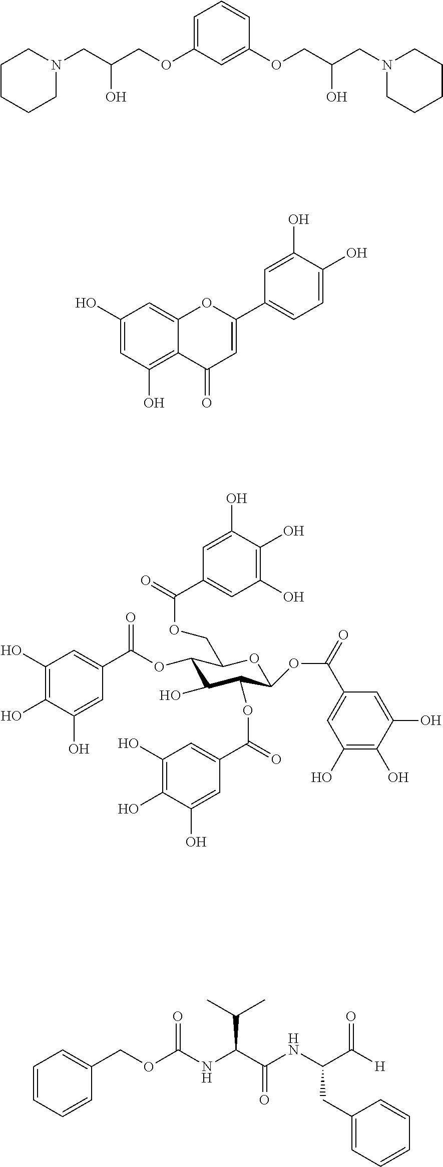

Remdesivir, Sofosbuvir, ribavirin, IDX-184 and GS-441524 have the following formulas:

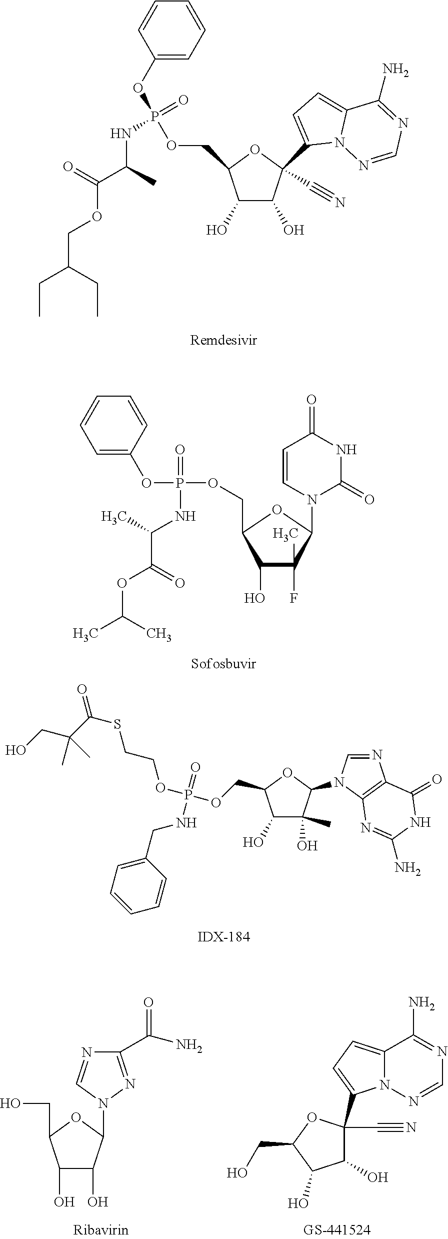

Additionally, one can administer compounds which inhibit the cytokine storm, such as dexamethasone, JAK inhibitors such as baricitinib, anti-coagulants and/or platelet aggregation inhibitors that address blood clots, or compounds which chelate iron ions released from hemoglobin by viruses such as COVID-19.

Representative ACE-2 inhibitors include sulfhydryl-containing agents, such as alacepril, captopril (capoten), and zefnopril, dicarboxylate-containing agents, such as enalapril (vasotec), ramipril (altace), quinapril (accupril), perindopril (coversyl), lisinopril (listril), benazepril (lotensin), imidapril (tanatril), trandolapril (mavik), and cilazapril (inhibace), and phosphonate-containing agents, such as fosinopril (fositen/monopril).

For example, when used to treat or prevent infection, the active compound or its prodrug or pharmaceutically acceptable salt can be administered in combination or alternation with another antiviral agent including, but not limited to, those of the formulae above. In general, in combination therapy, effective dosages of two or more agents are administered together, whereas during alternation therapy, an effective dosage of each agent is administered serially. The dosage will depend on absorption, inactivation and excretion rates of the drug, as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

A number of agents for monotherapy or combination therapy are disclosed in Ghosh et al., “Drug Development and Medicinal Chemistry Efforts Toward SARS-Coronavirus and Covid-19 Therapeutics,” ChemMedChem 10.1002/cmdc.202000223.

Nonlimiting examples of antiviral agents that can be used in combination with the compounds disclosed herein include those listed below.

### Compounds for Inhibiting the Cytokine Storm

Throughout its activation, the inflammatory response must be regulated to prevent a damaging systemic inflammation, also known as a “cytokine storm.” A number of cytokines with anti-inflammatory properties are responsible for this, such as IL-10 and transforming growth factor β (TGF-β). Each cytokine acts on a different part of the inflammatory response. For example, products of the Th2 immune response suppress the Th1 immune response and vice versa.

By resolving inflammation, one can minimize collateral damage to surrounding cells, with little or no long-term damage to the patient. Accordingly, in addition to using the compounds described herein to inhibit the viral infection, one or more compounds which inhibit the cytokine storm can be co-administered.

Compounds which inhibit the cytokine storm include compounds that target fundamental immune pathways, such as the chemokine network and the cholinergic anti-inflammatory pathway.

JAK inhibitors, such as JAK 1 and JAK 2 inhibitors, can inhibit the cytokine storm, and in some cases, are also antiviral. Representative JAK inhibitors include those disclosed in U.S. Pat. No. 10,022,378, such as Jakafi, Tofacitinib, and Baricitinib, as well as LY3009104/INCB28050, Pacritinib/SB1518, VX-509, GLPG0634, INC424, R-348, CYT387, TG 10138, AEG 3482, and pharmaceutically acceptable salts and prodrugs thereof.

HMGB1 antibodies and COX-2 inhibitors can be used, which downregulate the cytokine storm. Examples of such compounds include Actemra (Roche). Celebrex (celecoxib), a COX-2 inhibitor, can be used. IL-8 (CXCL8) inhibitors can also be used.

Chemokine receptor CCR2 antagonists, such as PF-04178903 can reduce pulmonary immune pathology.

Selective a7Ach receptor agonists, such as GTS-21 (DMXB-A) and CNI-1495, can be used. These compounds reduce TNF-α. The late mediator of sepsis, HMGB1, downregulates IFN-γ pathways, and prevents the LPS-induced suppression of IL-10 and STAT 3 mechanisms.

### Compounds for Treating or Preventing Blood Clots

Viruses that cause respiratory infections, including Coronaviruses such as Covid-19, can be associated with pulmonary blood clots, and blood clots that can also do damage to the heart.

The compounds described herein can be co-administered with compounds that inhibit blood clot formation, such as blood thinners, or compounds that break up existing blood clots, such as tissue plasminogen activator (TPA), Integrilin (eptifibatide), abciximab (ReoPro) or tirofiban (Aggrastat).

Blood thinners prevent blood clots from forming, and keep existing blood clots from getting larger. There are two main types of blood thinners. Anticoagulants, such as heparin or warfarin (also called Coumadin), slow down biological processes for producing clots, and antiplatelet aggregation drugs, such as Plavix, aspirin, prevent blood cells called platelets from clumping together to form a clot.

By way of example, Integrilin® is typically administered at a dosage of 180 mcg/kg intravenous bolus administered as soon as possible following diagnosis, with 2 mcg/kg/min continuous infusion (following the initial bolus) for up to 96 hours of therapy.

Representative platelet aggregation inhibitors include glycoprotein IIB/IIIA inhibitors, phosphodiesterase inhibitors, adenosine reuptake inhibitors, and adenosine diphosphate (ADP) receptor inhibitors. These can optionally be administered in combination with an anticoagulant.

Representative anti-coagulants include coumarins (vitamin K antagonists), heparin and derivatives thereof, including unfractionated heparin (UFH), low molecular weight heparin (LMWH), and ultra-low-molecular weight heparin (ULMWH), synthetic pentasaccharide inhibitors of factor Xa, including Fondaparinux, Idraparinux, and Idrabiotaparinux, directly acting oral anticoagulants (DAOCs), such as dabigatran, rivaroxaban, apixaban, edoxaban and betrixaban, and antithrombin protein therapeutics/thrombin inhibitors, such as bivalent drugs hirudin, lepirudin, and bivalirudin and monovalent argatroban.

Representative platelet aggregation inhibitors include pravastatin, Plavix (clopidogrel bisulfate), Pletal (cilostazol), Effient (prasugrel), Aggrenox (aspirin and dipyridamole), Brilinta (ticagrelor), caplacizumab, Kengreal (cangrelor), Persantine (dipyridamole), Ticlid (ticlopidine), Yosprala (aspirin and omeprazole).

Additional Compounds that can be Used

Additional compounds and compound classes that can be used in combination therapy include the following: Antibodies, including monoclonal antibodies (mAb), Arbidol (umifenovir), Actemra (tocilizumab), APN01 (Aperion Biologics), ARMS-1 (which includes Cetylpyridinium chloride (CPC)), ASC09 (Ascletis Pharma), AT-001 (Applied Therapeutics Inc.) and other aldose reductase inhibitors (ARI), ATYR1923 (aTyr Pharma, Inc.), Aviptadil (Relief Therapeutics), Azvudine, Bemcentinib, BLD-2660 (Blade Therapeutics), Bevacizumab, Brensocatib, Calquence (acalabrutinib), Camostat mesylate (a TMPRSS2 inhibitor), Camrelizumab, CAP-1002 (Capricor Therapeutics), CD24Fcm, Clevudine, (OncoImmune), CM4620-IE (CalciMedica Inc., CRAC channel inhibitor), Colchicine, convalescent plasma, CYNK-001 (Sorrento Therapeutics), DAS181 (Ansun Pharma), Desferal, Dipyridamole (Persantine), Dociparstat sodium (DSTAT), Duvelisib, Eculizumab, EIDD-2801 (Ridgeback Biotherapeutics), Emapalumab, Fadraciclib (CYC065) and seliciclib (roscovitine) (Cyclin-dependent kinase (CDK) inhibitors), Farxiga (dapagliflozin), Favilavir/Favipiravir/T-705/Avigan, Galidesivir, Ganovo (danoprevir), Gilenya (fingolimod) (sphingosine 1-phosphate receptor modulator), Gimsilumab, IFX-1, Ilaris (canakinumab), intravenous immunoglobulin, Ivermectin (importin a/β inhibitor), Kaletra/Aluvia (lopinavir/ritonavir), Kevzara (sarilumab), Kineret (anakinra), LAU-7b (fenretinide), Lenzilumab, Leronlimab (PRO 140), LY3127804 (an anti-Ang2 antibody), Leukine (sargramostim, a granulocyte macrophage colony stimulating factor), Losartan, Valsartan, and Telmisartan (Angiotensin II receptor antagonists), Meplazumab, Metablok (LSALT peptide, a DPEP1 inhibitor), Methylprednisolone and other corticosteroids, MN-166 (ibudilast, Macrophage migration inhibitory factor (MIF) inhibitor), MRx-4DP0004 (a strain of Bifidobacterium breve, 4D Pharma), Nafamostat (a serine protease inhibitor), Neuraminidase inhibitors like Tamiflu (oseltamivir), Nitazoxanide (nucleocapsid (N) protein inhibitor), Nivolumab, OT-101 (Mateon), Novaferon (man-made Interferon), Opaganib (yeliva) (Sphingosine kinase-2 inhibitor), Otilimab, PD-1 blocking antibody, peginterferons, such as peginterferon lambda, Pepcid (famotidine), Piclidenoson (A3 adenosine receptor agonist), Prezcobix (darunavir), PUL-042 (Pulmotect, Inc., toll-like receptor (TLR) binder), Rebif (interferon beta-la), RHB-107 (upamostat) (serine protease inhibitor, RedHill Biopharma Ltd.), Selinexor (selective inhibitor of nuclear export (SINE)), SNG001 (Synairgen, inhaled interferon beta-la), Solnatide, stem cells, including mesenchymal stem cells, MultiStem (Athersys), and PLX (Pluristem Therapeutics), Sylvant (siltuximab), Thymosin, TJM2 (TJ003234), Tradipitant (neurokinin-1 receptor antagonist), Truvada (emtricitabine and tenofovir), Ultomiris (ravulizumab-cwvz), Vazegepant (CGRP receptor antagonist or blocker), and Xofluza (baloxavir marboxil).

### Repurposed Antiviral Agents

A number of pharmaceutical agents, including agents active against other viruses, have been evaluated against Covid-19, and found to have activity. Any of these compounds can be combined with the compounds described herein. Representative compounds include lopinavir, ritonavir, niclosamide, promazine, PNU, UC2, cinanserin (SQ 10,643), Calmidazolium (C3930), tannic acid, 3-isotheaflavin-3-gallate, theaflavin-3,3′-digallate, glycyrrhizin, S-nitroso-N-acetylpenicillamine, favipivir, nelfinavir, niclosamide, chloroquine, hydroxychloroquine, 5-benzyloxygramine, ribavirin, Interferons, such as Interferon (IFN)-α, IFN-β, and pegylated versions thereof, as well as combinations of these compounds with ribavirin, chlorpromazine hydrochloride, triflupromazine hydrochloride, gemcitabine, imatinib mesylate, dasatinib, and imatinib.

### III. Preparation of Personalized Patient Reports

Patients suffering from a viral infection, such as a Coronaviridae infection, including infections by SARS-COV-2, may have different types of mutations in the viral genome. It can be useful to identify those mutations, and prepare a personalized medical treatment for the patient based on the type of virus, such as a Coronaviridae virus, and the mutations present in the virus.

In addition to obtaining sequencing information, one can input information from the patient, which can be stored in a first knowledge base, and which can include the sequencing information as well as additional patient information. Information on treatments for the particular type of Coronavirus, and particular mutations within that virus, can be stored in a second knowledge base. Expert rules for interpreting the data, and identifying effective therapies for patients with various mutations identified in the sequencing step, can be stored, for example, in a third knowledge base. Advisory data can be stored, for example, in a fourth knowledge base.

The presence of a single variant, or of multiple variants, can be correlated to effective therapy to treat the one variant or multiple variants. Each variant, and its corresponding mutations, can be analyzed against the knowledge base of therapeutic agents and the knowledge base of expert rules for determining which of the therapies is effective against the particular mutations in the variants, and appropriate therapy to treat all of the variants can be determined.

The report may include a listing of the types of variants, as well as the therapies that will work against these variants, and, optionally, therapies that will not work against these variants. Optionally, the report can also include advisory information.

The type of patient information that may be obtained, and how the various knowledge bases are set up and managed, is described below. Also described below are the types of systems and software used to manage the data, as well as the types of reports that can be generated.

The present invention is described below with reference to flowchart illustrations of methods, apparatus (systems), and computer program products according to an embodiment of the methods described herein. It will be understood that each block of the flowchart illustrations, and combinations of blocks in the flowchart illustrations, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions specified in the flowchart block or blocks.

The methods described herein, as well as the system and software used to implement the methods, enable one to guide the decision, or to optimize the decisions, whether or not to perform sequencing (such as Sanger sequencing) on a given sample, based on the patient's information and interpretation by the system.

### Patient Information

Patient information is ideally entered into a system, which can then use the information to determine an appropriate treatment regimen. The information includes, at least, sequencing information, which identifies major and, optionally, minor variants of the types of Coronaviridae, and, optionally, other as viruses (including HIV, HBV, and HCV) with which the patient is infected, and the specific mutations on each of these variants. Such information is useful, particularly in the treatment of Coronaviridae infections, because there is a significant difference between two or more mutations on a single virus, or different mutations on different viruses. This is particularly relevant with antiviral therapies, where the presence of a single mutation can be associated with failure of a first treatment modality, but the presence of an additional mutation can be associated with the renewed effectiveness of this treatment modality. That is, drugs which are inactive against virus with a first mutation may be active against virus with a first and a second mutation. Without knowing whether a particular combination of mutations occurs on a single variant, or on multiple variants, it can be difficult to design appropriate therapy. Because the methods described herein can provide information on which mutations are present in which variants, appropriate therapeutic modalities can be prescribed.

In one embodiment, after entering the patient's genetic information (i.e., types of variants, and mutations present on each variant), a user-defined therapeutic treatment regimen for the disease (or medical condition) can be entered. Advisory information for the user-defined combination therapeutic treatment regimen can then be generated. Where a rejected therapeutic treatment regimen for the disease (or medical condition) is entered, for example, a regimen that is included in the knowledge base of therapeutic regimens, but not recommended (i.e., given a very low ranking), advisory information can be generated, providing one or more reasons for not recommending (or providing a low ranking) for the particular therapeutic treatment regimen.

Additional examples of patient information that may be gathered include one or more of co-morbidities known to result in a higher likelihood of hospitalization (such as diabetes, obesity, anxiety, and the like), gender, age, weight, viral load information, virus genotype and phenotype information, hemoglobin information, neuropathy information, neutrophil information, pancreatitis, hepatic function, renal function, drug allergy and intolerance information, and information for drug treatments for other conditions. The information may include historical information on prior therapeutic treatment regimens for other diseases or medical conditions with which the patient is suffering. This can be particularly important where, as is the case with SARS-CoV-2, the vast majority of patients with mortality or significant morbidity are those with four or more co-morbidities.

While the patient is typically examined on a first visit to determine the patient information, it will be appreciated that patient information may also be stored in the computing device, or transferred to the computing device from another computing device, storage device, or hard copy, when the information has been previously determined.

### Expert Rules/Algorithms, Knowledge Base Management, and Computer Hardware/Software

Some embodiments of the methods described herein are described below with reference to flowchart illustrations of methods, apparatus (systems), and computer program products. It will be understood that each block of the flowchart illustrations, and combinations of blocks in the flowchart illustrations, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

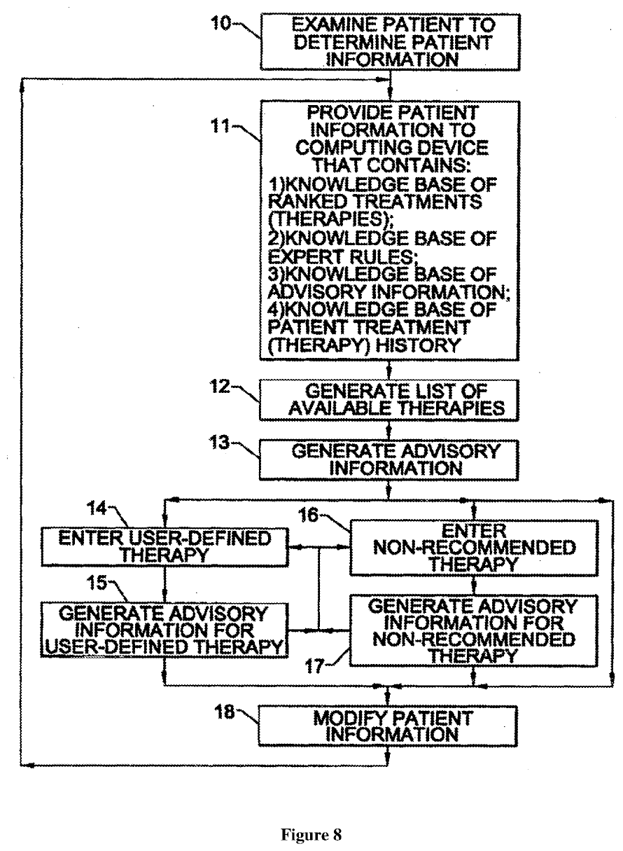

One embodiment of the methods described herein is illustrated in FIG. 8. In the first step 10, a crystal structure of an enzyme, such as the protease 3CLpro, complexed with a protease inhibitor, is evaluated in silico using the predictive model described herein to identify mutations that cause the protease inhibitor to have a significantly lower binding affinity for the protease. In the second step 20, crystal structures of the enzyme and one or more the substrates the enzyme complexes with when its activity is not being inhibited are evaluated using the predictive model described herein. This identifies mutations from step 10 that also result in significantly lower binding of the enzyme to the one or more substrates. Mutations identified in the first step 10 that do not result in significantly lower binding of the enzyme to the substrate(s) are predicted to be mutations associated with drug resistance. The mutations associated with drug resistance are stored in Database 1 (30), and the process is repeated for other drugs that are inhibitors of the enzyme, for which crystal structures of the enzyme complexed with the inhibitors are available. This builds a database (30) of known mutations associated with treatment with known inhibitors.

From this database (30), one can prepare a second database (40) where mutations associated with a first inhibitor are used to screen one or more other inhibitors, to identify inhibitors that would bind the enzyme if it mutated to avoid the first inhibitor. This identifies potential treatments that would be effective if the virus infecting a given patient had one type of mutation that indicated treatment with the first inhibitor would likely be ineffective. This process can be repeated with as many inhibitors as there are crystal structures of the inhibitors and the enzyme. The potentially-effective treatments are stored in the second database (40), optionally along with other treatments that do not involve inhibition of the particular enzyme.

The mutations associated with drug resistance are mutations in the protein sequence. These mutated protein sequences can be used to create a library of primers associated with the DNA that encodes the mutated protein sequences. A third database (50) can be prepared which correlates the presence of DNA or RNA that binds to one or more of these primers to resistance to an inhibitor that does not bind to a virus with the mutation associated with the primer.

A biological sample taken from one or more infected patients can be screened, for example, using Sanger sequencing, to identify which mutations associated with drug resistance are present in the virus, and this information inputted into a computer system that compares the mutations with those in the third database (50). This identifies the particular viral variant with which the patient is infected. The viral variant can be cross-referenced with the treatments stored in the second database (40) to identify potentially effective treatments for each patient.

In the event there are no effective treatments associated with inhibitors of the enzyme, due to the existence of sufficient mutations that none of the evaluated inhibitors would be expected to be effective, then additional treatment regimens using active agents other than inhibitors of this particular enzyme can be evaluated. In some embodiments, the additional treatment regimens are also present in the second database (40), such that an effective treatment can be identified with the second database (40) even if none of the evaluated inhibitors are effective.

In some embodiments, a fourth database (60) can include expert rules, prepared using the experience of treating physicians based on the successful treatment of patients with the same or similar variants, optionally with a ranking system to identify the potential treatments in order of their likelihood of success with a given variant.

In some aspects of these embodiments, combination therapies that are likely to be effective, without a substantial risk of producing new variants, much like HAART is used for HIV, can be included in the database, based on the expert rules and experiences of the physicians who created the information on effective treatments used to prepare the expert rules.

In other aspects of these embodiments, the expert rules can identify treatment regimens that are not recommended for patients with various co-morbidities. Where information on a patient's co-morbidities, as well as their particular viral variant, is entered into a program that correlates mutations with potentially effective treatments, the list of potentially effective can be compared with treatments that are not suggested if a patient has certain co-morbidities. By removing therapies that might be effective against a given viral variant, but are incompatible with one or more of a patient's comorbidities, one or more potentially effective treatment regimens can be identified that are compatible with the patient's co-morbidities and the particular variant with which the patient is infected.

In other aspects of these embodiments, the expert rules can identify treatment regimens that are not recommended for patients which take certain medications, such as metformin for treating diabetes. A program can be used to compare potentially effective treatment regimens to identify those which are incompatible with other drugs the patient is taking for other indications. Where one or more of the medications are incompatible with one or more suggested treatment regimens, from the list of available treatment regimens which are predicted to be effective with the particular viral variant with which the patient is infected, such treatment regimens can be removed from the list of potentially effective treatment regimens. When a personalized report is prepared, it will be limited to potentially effective treatment regimens that are compatible with other medications the patient is taking.

Thus, in some embodiments, information on a patient's co-morbidities and/or other medications the patient is taking, as well as their particular viral variant, is entered into a program that correlates mutations with potentially effective treatments. The list of potentially effective treatments can be compared with treatments that are not suggested if a patient has certain co-morbidities or takes certain medications. By removing therapies that might be effective against a given viral variant, but are incompatible with one or more of a patient's comorbidities and/or one or more medications the patient is taking, one or more potentially effective treatment regimens can be identified that are compatible with the patient's co-morbidities, the medications the patient is taking, and the particular variant with which the patient is infected.

Thus, the information stored in the various databases, for example, the second and/or fourth databases, can be used to prepare a personalized report for each patient outlining potential treatment regimens that would be expected to be effective.

In some embodiments, the patient information that may be gathered include one or more of gender, age, weight, viral load information, information on viral variants, hemoglobin information, optionally including the results of a d-dimer test to determine whether the patient has significant blood clotting, neuropathy information, neutrophil information, pancreatitis, hepatic function, renal function, drug allergy and intolerance information, and information for drug treatments for other conditions. The information may include historical information on prior therapeutic treatment regimens for the disease or medical condition.

While the patient is typically examined on a first visit to determine the patient information, it will be appreciated that patient information may also be stored in the computing device, or transferred to the computing device from another computing device, storage device, or hard copy, when the information has been previously determined.

The patient information can then be provided to a computing device that contains a knowledge base of treatments (i.e., one or more of the databases described above), contains a knowledge base of expert rules for determining available treatment options for the patient in light of the patient information, and also contains a knowledge base of advisory information. A list of available treatments for the patient is then generated from the patient information and the available treatments by the expert rules, and advisory information for the available treatments is generated. The advisory information may include warnings to take the patient off a contraindicated drug or select a suitable non-contraindicated drug to treat the condition before initiating a corresponding treatment regimen and/or information clinically useful to implement a corresponding therapeutic treatment regimen.

### Computer Program Instructions

The computer program instructions described herein can be stored in a computer-readable memory that can direct a computer or other programmable data processing apparatus to function in a particular manner, such that the instructions stored in the computer-readable memory produce an article of manufacture including instruction means which implement the function specified in the flowchart block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

One embodiment of the diagnostic/treatment methods (i.e., theranostic methods) described herein is illustrated in FIG. 8. In the first step 10, the patient is examined to determine patient information. The patient information is then provided 11 to a computing device that contains a knowledge base of treatments, contains a knowledge base of expert rules for determining available treatment options for the patient in light of the patient information, and also contains a knowledge base of advisory information. A list of available treatments for the patient is then generated 12 from the patient information and the available treatments by the expert rules, and advisory information for the available treatments is generated 13. The advisory information may include warnings to take the patient off a contraindicated drug or select a suitable non contraindicated drug to treat the condition before initiating a corresponding treatment regimen and/or information clinically useful to implement a corresponding therapeutic treatment regimen.

For example, when the known disease is a Coronaviridae infection, the treatment regimen includes antiviral drugs, and the treatment regimen or advisory information may also include contraindicated or potentially adversely interacting non-antiviral drugs. Particularly, when the treatment regimen includes a protease inhibitor, a contraindicated drug may be terfenadine. When the treatment regimen includes indinavir, a contraindicated drug is cisapride. Exemplary antiviral drugs, particularly ones useful for treating a Coronaviridae infection, are described in detail above.

An “inference engine” can be used to process one or more potential therapies from a Therapies resource file which contains one or more valid therapies, and may support multiple drug output data combinations. Those therapies which are recommended by types below the knowledge base may be displayed. Commentaries, which include warnings and/or advisories concerning drugs as well as various patient conditions, can also be provided. Commentaries may appear in specific locations of the User Interface. Commentaries may have various Flags, Triggers, and Output Locations.

Rejection Notices can be provided, which provide explanation why a given therapy is not recommended.

The base dosage and any adjustments to the base dosage due to various patient conditions may also be calculated by the inference engine. This information may also include the number of pills in the therapy, and the number of times the patient will be taking medications for a given therapy. For a multi-drug therapy, the frequency of the therapy is the drug in the therapy that has the highest number of Frequencies. If a three-drug regimen has 2 drugs with q12h dosages and one that is a q8h, the therapy is considered to be a q8h Frequency.

In terms of relative efficacy, based on potentially-effective treatments and the experience of treating physicians as articulated in the expert rules, an adjusted score can be prepared based on the therapy that is predicted to be the most effective, followed by therapies that are predicted to be somewhat less effective. This can help provide a treating physician with the most optimal treatment regimen.

In one representative, non-limiting example, the system evaluates a therapy containing a drug that is known to be associated with a medical condition in that patient's medical history, therefore the therapy is ranked low, as it would be less likely to be successful given the patient's specific history and characteristics. Each potentially-effective therapy can have a starting efficacy rating, which reflects the therapy's anticipated relative efficacy score, and this relative efficacy score can then be adjusted up or down by the rules.

The inference engine may process one or more potential therapies stored in the databases, for example, those stored in a Therapies resource file, and may process every therapy included in this file. Commentaries consist of warnings and advisories concerning drugs as well as various patient conditions. An individual patient report can include such Commentaries, including various flags, triggers, and warnings. Optionally, Rejection Notices can be used to explain why a given therapy is not recommended. Such Rejection notices may appear in predefined places in a particular patient report.

The “Adjusted Score” may be based on patient specific characteristics to roughly indicate the likelihood of that therapy being an effective treatment for that patient. An example would be: the system evaluates a therapy containing a drug that is known to be associated with a medical condition in that patient's medical history, therefore the therapy is ranked low. Each potentially-effective therapy can have a starting number (i.e., the therapy's relative efficacy score), which can then be adjusted up or down by the rules. In some embodiments, the patient report includes both the base “Efficacy” number and the “Adjusted Score” number, based on the patient's comorbidities and/or other medications the patient is taking.

Comorbidities that may adversely affect patient outcomes include, but are not limited to, cardiovascular disease (including but not limited to congestive heart failure, hypertension, hyperlipidemia and angina), pulmonary disease (including but not limited to chronic obstructive pulmonary disease, asthma, pneumonia, cystic fibrosis, and tuberculosis), neurologic disease (including but not limited to Alzheimer's disease, Parkinson's disease, epilepsy, multiple sclerosis, amyotrophic lateral sclerosis or ALS, psychoses such as schizophrenia and organic brain syndrome, neuroses, including anxiety, depression and bipolar disorder), hepatitis infections (including hepatitis B and hepatitis C infection), urinary tract infections, venereal disease, cancer (including but not limited to breast, lung, prostate, and colon cancer), etc.

The predictive methods described herein can be useful for known viral diseases where there is a crystal structure of an enzyme, such as a protease or polymerase, in complex with an inhibitor of that enzyme, and a crystal structure of the enzyme in complex with the substrate(s) with which it interacts. Representative examples include all Coronaviridae, including SARS-COV-2, HIV, and hepatitis viruses. The predictive methods can be used for infections in which mono-therapy is commonly used, and for infections in which combination therapy is commonly used.

Advantageously, the list of available treatments and advisory information may be regenerated in a number of ways. The patient information may be simply modified. In addition, if a particular therapy in which the user might be interested is not presented, a user-defined therapy may be entered and advisory information generated based on the user-defined therapy. Still further, if a therapeutic treatment regimen that is in the knowledge base is rejected by the system (not recommended upon display), the non-recommended therapeutic treatment regimen may be entered and advisory information generated for the non-recommended therapeutic treatment regimen. This may indicate to the user that they should discontinue use of a non-critical drug for another condition or select a suitable substitute that does not create a conflict/non-recommended situation so that they can then proceed with the therapy of choice. Alternatively, the advisory information can be generated automatically for non-recommended therapeutic treatment regimens. These various steps can be repeated in any sequence in an interactive manner to provide the user with assurance that all treatment options have been given adequate and appropriate consideration.

The terms “therapy” and “therapeutic treatment regimen” are interchangeable herein and, as used herein, mean any pharmaceutical or drug therapy, regardless of the route of delivery (e.g., oral, intraveneous, intramuscular, subcutaneous, intraarterial, intraperitoneal, intrathecal, etc.), for any disease (including both chronic and acute medical conditions, disorders, and the like). In addition, it is understood that the present invention is not limited to facilitating or improving the treatment of diseases. The present invention may be utilized to facilitate or improve the treatment of patients having various medical conditions, without limitation.

### System Description

As discussed above, once mutations predicted to be associated with the administration of a particular drug or drugs are mapped out, and a patient infected with and the virus is screened for the presence of these mutations, appropriate therapy can be selected. The combination of diagnosis and therapy is known as a theranostic method. The theranostic methods described herein may be embodied as an expert system that provides decision support to physicians (or other health care providers) treating patients with a known disease, such as Coronaviridae infection. A system according to the present invention calculates appropriate antiviral therapy options and can attaches relevant information, such as expert information, to those options.

As known to those of skill in the art, an expert system, also known as artificial intelligence (AI), is a computer program that can simulate the judgment and behavior of a human or an organization that has expert knowledge and experience in a particular field. An expert system typically contains a knowledge base containing accumulated experience and a set of rules for applying the knowledge base to each particular situation that is described to the program. Expert systems are well known to those of skill in the art and need not be described further herein.

The antiviral therapy options (including monotherapy, or combinations of antiviral drugs), are derived using a knowledge base consisting of a number of expert system rules and functions which in turn take into account a given patient's treatment history, current condition and laboratory values. In some embodiments, a system as described herein can support the entry, storage, and analysis of patient data in a large central database. The system can have a flexible data-driven architecture and custom reporting capabilities designed to support patient therapy management and clinical drug trial activities such as screening, patient tracking and support. It is anticipated that a system described herein may be used by health care providers (including physicians), clinical research scientists, and possibly healthcare organizations seeking to find the most cost-effective treatment options for patients while providing the highest standard of care.

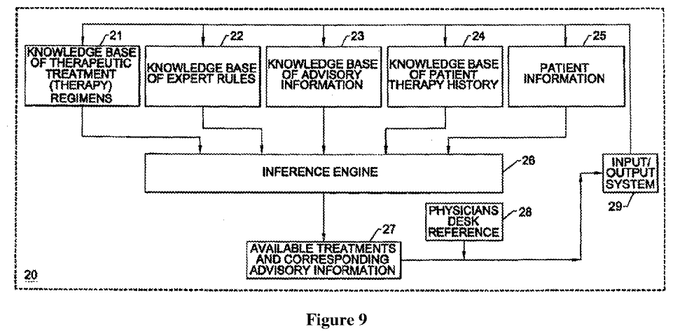

A system for carrying out the theranostic methods described herein is schematically illustrated in FIG. 9. The system 20 comprises a knowledge base of treatment regimens 21, which may be ranked for efficacy (e.g., by a panel of experts) or ranked according to system rules, a knowledge base of expert rules 22, a knowledge base of advisory information 23, a knowledge base of patient therapy history 24 and patient information 25. Patient information is preferably stored within a database and is configured to be updated. The knowledge bases and patient information 21-25 may be updated by an input/output system 29, which can comprise a keyboard (and/or mouse) and video monitor. Note also that, while the knowledge bases and patient data 21-25 are shown as separate blocks, the knowledge bases and patient data 21-25 can be combined together (e.g., the expert rules and the advisory information can be combined in a single database).

To conduct the theranostic methods described above, the information from blocks 21-25 is provided to an inference engine 26, which generates the listing of available treatments and the corresponding advisory information from the information provided by blocks 21-25. The inference engine 26 may be implemented as hardware, software, or combinations thereof. Inference engines are known and any of a variety thereof may be used to carry out the present invention. Examples include, but are not limited to, those described in U.S. Pat. No. 5,263,127 to Barabash et al. (Method for fast rule execution of expert systems); U.S. Pat. No. 5,720,009 to Kirk et al. (Method of rule execution in an expert system using equivalence classes to group database objects); U.S. Pat. No. 5,642,471 to Paillet (Production rule filter mechanism and inference engine for expert system); U.S. Pat. No. 5,664,062 to Kim (High performance max-min circuit for a fuzzy inference engine).

High-speed inference engines are preferred so that the results of data entered are continually updated as new data is entered. As with the knowledge bases and patient information in blocks 21-25, the inference engine 26 may be a separate block from the knowledge bases and patient information blocks 21-25, or may be combined together in a common program or routine.

Note that the advisory information that is generated for any available therapy may differ from instance to instance based on differences in the patient information provided.

### System Architecture

The prediction of effective therapies can be implemented as a system running on a stand-alone computing device. In one embodiment, it is implemented as a system in a client-server environment. As is known to those of skill in the art, a client application is the requesting program in a client-server relationship. A server application is a program that awaits and fulfills requests from client programs in the same or other computers. Client-server environments may include public networks, such as the Internet, and private networks often referred to as “intranets”, local area networks (LANs) and wide area networks (WANs), virtual private networks (VPNs), frame relay or direct telephone connections. It is understood that a client application or server application, including computers hosting client and server applications, or other apparatus configured to execute program code embodied within computer usable media, operates as means for performing the various functions and carries out the methods of the various operations of the present invention.

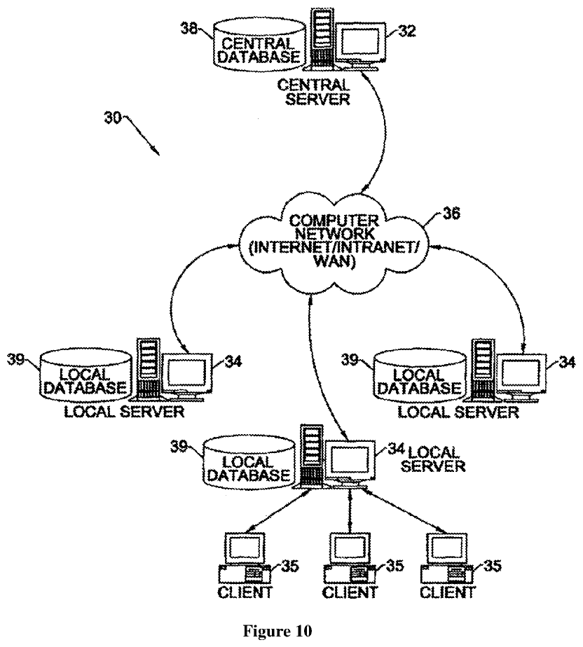

Referring now to FIG. 10, a client-server environment 30 according to a preferred embodiment of the present invention is illustrated. The illustrated client-server environment 30 includes a central server 32 that is accessible by at least one local server 34 via a computer network 36, such as the Internet. A variety of computer network transport protocols including, but not limited to TCP/IP, can be utilized for communicating between the central server 32 and the local servers 34.

### Central Server

The central server 32 includes a central database 38, such as the Microsoft® SQL Server application program, version 6.5 (available from Microsoft, Inc., Redmond, Wash.), executing thereon. The central server 32 ensures that the local servers 34 are running the most recent version of a knowledge base. The central server 32 also stores all patient data and performs various administrative functions including adding and deleting local servers and users to the system (20, FIG. 2). The central server 32 also provides authorization before a local server 34 can be utilized by a user. Patient data is preferably stored on the central server 32, thereby providing a central repository of patient data. However, it is understood that patient data can be stored on a local server 34 or on local storage media.

### Local Server

Each local server 34 typically serves multiple users in a geographical location. Each local server 34 includes a server application, an inference engine, one or more knowledge bases, and a local database 39. Each local server 34 performs artificial intelligence processing for carrying out operations of the present invention. When a user logs on to a local server 34 via a client 35, the user is preferably authenticated via an identification and password, as would be understood by those skilled in the art. Once authenticated, a user is permitted access to the system (20, FIG. 9) and certain administrative privileges are assigned to the user.

Each local server 34 also communicates with the central server 32 to verify that the most up-to-date version of the knowledge base(s) and application are running on the requesting local server 34. If not, the requesting local server 34 downloads from the central server 32 the latest validated knowledge base(s) and/or application before a user session is established. Once a user has logged onto the system (20, FIG. 9) and has established a user session, all data and artificial intelligence processing is preferably performed on a local server 34. An advantage of the illustrated client-server configuration is that most of the computationally intensive work occurs on a local server 34, thereby allowing “thin” clients 35 (i.e., computing devices having minimal hardware) and optimizing system speed.

In one embodiment, each local server database 39 is implemented via a Microsoft® SQL Server application program, Version 6.5. The primary purpose of each local database 39 is to store various patient identifiers and to ensure secure and authorized access to the system (20, FIG. 9) by a user. It is to be understood, however, that both central and local databases 38, 39 may be hosted on the central server 32.

### Local Client

Each local client 35 also includes a client application program that consists of a graphical user interface (GUI) and a middle layer program that communicates with a local server 34. Program code for the client application program may execute entirely on a local client 35, or it may execute partly on a local client 35 and partly on a local server 34. As will be described below, a user interacts with the system (20, FIG. 9) by entering (or accessing) patient data within a GUI displayed within the client 35. The client 35 then communicates with a local server 34 for analysis of the displayed patient information.

Computer program code for carrying out operations of the present invention is preferably written in an object oriented programming language such as JAVA®, Smalltalk, or C++. However, the computer program code for carrying out operations of the present invention may also be written in conventional procedural programming languages, such as the “C” programming language, in an interpreted scripting language, such as Perl, or in a functional (or fourth generation) programming language such as Lisp, SML, or Forth.

The middle layer program of the client application includes an inference engine within a local server 34 that provides continuous on-line direction to users, and can instantly warn a user when a patient is assigned drugs or a medical condition that is contraindicated with, or antagonistic of, the patient's current antiretroviral therapy. Every time patient data is entered into the system (20, FIG. 9) or updated, or even as time passes, the inference engine evaluates the current status of the patient data, sorting, categorizing, ranking and customizing every possible antiretroviral therapy for a patient according to the specific needs of the patient.

### Inference Engine

Inference engines are well known by those of skill in the art and need not be described further herein. Each knowledge base used by an inference engine is a collection of rules and methods authored by a one or more physicians and scientists who treat a particular type of viral infection. A knowledge base may have subjective rules, objective rules, and system-generated rules. Objective rules can be used to correlate a drug with a low probability of success due to the presence of one or more mutations associated with drug resistance. Objective rules can also include industry-established facts regarding the treatment of the particular viral disorder, and can include information drawn from package insert information for drugs used to treat the disorder.

For objective rules, the system can be configured so as to prevent a user from receiving recommendations on new therapy options when certain crucial data on the patient has not been entered. However, it is understood that this does not prevent a health care provider, such as a physician, from recording his/her therapy decisions, even if the system (20, FIG. 9) has shown reasons why that therapy may be harmful to the patient. That is, the health care provider is typically the final authority regarding patient therapy.

Subjective rules can be based, for example, on expert opinions, observations and experience. Subjective rules are typically developed from “best practices” information based on consensus opinion of experts in the field. Such expert opinion may be based on knowledge of the literature published or presented in the field or their own experience from clinical practice, research or clinical trials of approved and unapproved medications. Ideally, a number of experts are used so that personal bias is reduced.

System generated rules are those derived from the outcomes of patients tracked in the system who received known and defined therapies and either improved, stabilized or worsened during a defined period. Because of the large number of potential combinations usable in treating viral infections, this system generated database and rules derived from them are likely to encompass data beyond that achievable from objective or subjective rules databases.

The rules which comprise the various knowledge bases (21-24, FIG. 9) each have two main parts: a premise and a conclusion—also referred to as the left side and the right side, respectively. When a premise of a rule is found to be true, the action specified in the conclusion is taken. This is known to those of skill in the art as “firing” the rule. For example, consider the following rule, with respect to the treatment of SARS-COV-2:

If the patient sample include a S144W or S144Y mutation, then do not administer the protease inhibitor PF07321332.

The premise of the above rule is for the inference engine to determine whether or not a therapy being evaluated (i.e., “eval therapy”) contains the protease inhibitor PF07321332. If a therapy does contain protease inhibitor PF07321332, the action called for by the conclusion of the rule is to attach the commentary to the therapy. The commentary may be a piece of text that provides a user with the necessary information about therapies containing protease inhibitor PF07321332.

Representative types of commentary include:

Rules that provide information on therapy change or initiation Boundary condition rules: Limits for values, intervals for values to be updated Comment Data Aging rules:

These rules warn the user that the data in certain fields is getting old and that the most current values in the system will be used.
Rules that filter therapies due to drug interactions in drug combinations
Rules that filter therapies due to medical conditions
Rules that filter therapies due to genotypic mutations in patient's virus
Rules that filter therapies due to phenotypic sensitivity/resistance
Therapy ranking rules
General dosage rules
Solid dosage rule
Dosage modifications due to various drug combinations
Dosage modification due to medical conditions
Commentaries added due to medical conditions
Commentaries added due to drug interactions
Commentaries added due to drug combination

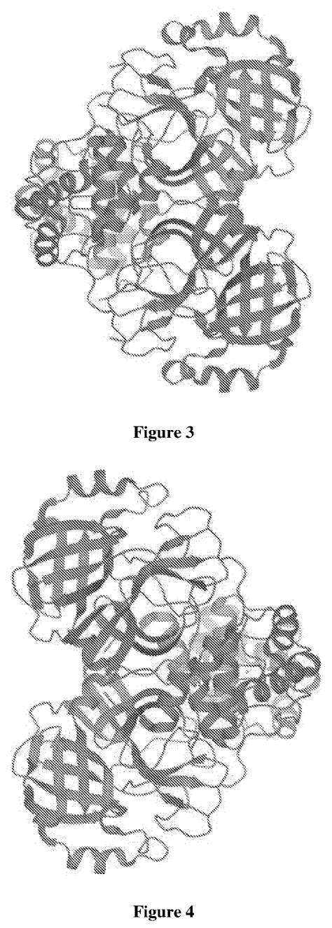

Using the various knowledge bases and patient information of the present invention (21-25, FIG. 9), the inference engine (26, FIG. 9) can evaluate potential therapy options for a patient based on a patient's medical history (including therapy history) and current laboratory values. FIG. 3 shows a client-server environment within which the system of FIG. 9 can operate. A central server (32) with a central database (38) is connected via a computer network (36), such as an internet, intranet, or wide area network (WAN), which is connected to local servers (34), which include local databases (39), which can be accessed by clients (35). Multiple antiretroviral drug combinations can be quickly and accurately analyzed for a particular patient. Furthermore, the inference engine can quickly provide guidance in the areas listed below.

Are there conflicts between lab data which indicates resistance to one or more drugs in the patient's current therapy and current viral load data which indicates significant viral suppression?

Should antiviral therapy be initiated for the patient?

Is the patient's current therapy achieving good initial and long-term viral suppression or should the therapy be changed?

Are there potential non-compliance issues as demonstrated by a lack of viral suppression with a regimen when current genotype or phenotype data does provide explanation for the failure by demonstrating resistance to any drugs in the patient current therapy?

What are the base and adjusted dosages of antiretroviral drugs in a given therapy? Are there any special specific dosage administration instructions?

Which antiretroviral drugs can be used with indications each other and what dosage adjustments are required?

Are there any contraindications or interactions between antiretroviral drugs in patient's current therapy or potential therapies and the non-antiretroviral drugs patient is taking and if so what are they and what, if any, dosage adjustments are required?

Are there any medical conditions to be aware of in deciding an appropriate therapy for patient?

What, if any, effect do current or historical medical conditions have on each therapy option?

What is the dosing frequency of the drugs in the therapy? What are all the drug combination therapy options for the patient?

What information from the package inserts from each drug apply specifically to the patient? What is the relative antiviral efficacy of each therapy?

Are there special considerations that might make one therapy more or effective for patient? To what drugs are the patient's virus current genotypic or phenotypic profile known to be associated with resistance?

Which antiviral drugs are more effective against resistant strains when used together? Which drugs (if any) used in historical therapies are most likely to be effective if recycled into a new therapy?

Can any of the drugs in patient's current therapy be recycled into the next therapy?

### User Interface

A medical history user interface can be used to enter data about a patient's medical history. The user interface allows a user to create, save, update and print patient records. When a user adds a new patient, the medical history user interface appears with empty data entry fields. Data entry fields for receiving information via a GUI are well known to those of skill in the art and need not be described further herein. When a user opens a patient record for editing, the medical history user interface appears with patient data in the various fields. Color can optionally be used to highlight critical or required information in a patient record.

Representative elements in a medical history user interface can include a “print” button, for printing a patient record and therapeutic treatment regimen details; a “save” button for saving a patient record; and a “speed entry” check box for allowing a user to move quickly between entry fields. In addition, group headings can be used to divide a patient's medical history into related categories. An “add” button can allow a user to add new information to a patient record for a selected group. A “delete” button can allow a user to delete patient information for a selected group (although the original information may still remain recorded in the database). A “history” button can allow a user to review a patient's historical data for each selected group.

After completing a patient's medical history, an inference engine can analyze the data and suggest whether a therapeutic treatment regimen is indicated, if an existing therapeutic treatment regimen should be continued or changed, and the best drug therapies for the selected patient. Often, more than one drug therapy is presented to the user. These drug therapies are preferably ranked according to expected efficacy, frequency in dosage, pill count, and cost. All of these factors can help the user make a decision about what therapy to use for the selected patient. In some embodiments, when a user clicks on a drug therapy in the presented list, information is provided about the dosage regimens. Also, various warnings, such as drug interaction warnings, and notes about each drug, may be presented. An appropriate drug therapy can then be selected.

A list of available antiviral drugs can optionally displayed. A user desiring to evaluate a particular combination of drugs can click the appropriate check boxes to review information in a “therapy details” box. A “Use as Current Therapy” button can allow a user to apply a particular therapy to a patient. Various hyperlinks can be used within a “therapy details” box allow a user to display specific information about a therapy evaluation. For example, a user can be allowed to view a rule which is associated with the displayed text.

In some embodiments, a “resistance evaluation alert” can be provided adjacent each available antiviral drug displayed within the box. For example, an icon or other flag can be used to indicate that a patient's last genotype test contains mutations which are known to be associated with full or partial resistance to the antiviral drug, or that a patient's last phenotype test demonstrated resistance to the antiretroviral drug.

Within a “therapy list” box, various symbols can be used to provide information about a drug therapy option. These symbols provide an instant graphical warning level for each therapy option. Some symbols, such as a red exclamation point, can be used to indicate that there is critical, possibly life threatening information in the therapy details box for that therapy which must be read in order for that therapy to be properly used.

When a drug therapy is selected by a user for evaluation, a “therapy details” box can be displayed in “full screen” mode. Representative elements to include in an illustrated “therapy details” box include an identification box for identifying the therapy being evaluated; a “Use as Current Therapy” button that allows a user to apply a particular therapy to a patient; and a “Show Therapies” button that returns the therapy details box back to half-screen size. In addition, various hyperlinks may be embedded within text displayed within the therapy details box that can be activated by a user to display various types of information. Alert banners can be displayed at the top of the therapy details box 73 if alerts are to be used. Dosages of each drug, along with special administration instructions, can be displayed within the therapy details box. Dosage adjustment information and various warnings and advisories can also be displayed within the therapy details box. In some embodiments, therapeutic treatment regimens are not displayed to a user if an invalid drug (i.e., one that is expected to be ineffective, or contraindicated due to a patient's medical history or other drugs the patient is taking) is selected for treatment of a patient.

### Physicians Desk Reference®

In some embodiments, the Physicians Desk Reference® (PDR®) is fully integrated with the system 20 of FIG. 9. Users can access the drug abstracts for antiviral drugs listed in the therapy list box of the therapy evaluation user interface. In addition, users can access the PDR® on-line Web database to obtain additional information about a specific drug or to research a substitute for a contraindicated drug. When a user selects a drug within the therapy list box of the therapy evaluation user interface, a web browser can optionally be launched and the PDR® on-line Web database can be accessed. Information can also be extracted from the PDR® on-line Web database to provide drug selection lists for non-antiviral drugs that a patient may be taking and to define relationships between brand name and generic drugs.

It is important to validate the information that is obtained, to ensure that it is accurate. The following sections discuss validation of the information obtained during the screening of patient samples.

### Data Entry Quality Assessment

In one aspect of this embodiment, for each type of protein (for example, where a patient is screened for the presence of mutations in a viral enzyme, the proteins can include reverse transcriptase, protease, polymerase, integrase, GP120, and GP41), the list of parameters to be used can be fully customizable through a dedicated interface.

When assessing the quality of the assessment of a patient's particular viral mutations, sequence quality assessment can be performed at the reads level. Specific visualization, editing, filtering interfaces can be applied, to work on the reads. One or more types of filters can be used, for example, a homopolymer check at positions of interest.

### Use of the Methods to Monitor a Patient's Progress

By following a patient's progress over time, one can also obtain information about the efficacy of previous treatment regimens imposed on patients, including one or more of the viral load, the development of mutations, the development of side effects, and the like.

### Use of the Methods in Research

In addition to being used for routine genotyping, the process can also be used for research, for example, to identify types of mutations in a pathogen and/or in the host following the administration of particular anti-viral agents. The system can be interfaced with a dedicated Data Exploratory Framework that can be used for research, either on sequence data only, or in combination with clinical data.

The present invention will be better understood with reference to the following non-limiting examples.

### Examples

### Example 1: Analysis of Potential Mutations on 3CLpro

Coronaviruses like SARS-COV-2 include a 3C-like protease (3CL or 3CLpro) enzyme. The wild-type SARS-COV-2 3CLpro is described, for example, in Muhammad Tahir ul Qamar, et al., “Structural basis of SARS-COV-2 3CLpro and anti-COVID-19 drug discovery from medicinal plants,” Journal of Pharmaceutical Analysis, Volume 10, Issue 4, 2020, Pages 313-319, ISSN 2095-1779, https://doi.org/10.1016/j.jpha.2020.03.009. The sequence for this protease is found at GenBank accession no. AY609081.1. The amino acid sequence from GenBank accession no. AY609081.1 is provided below, with the first five amino acids truncated:

SGFRK MAFPSGKVEG CMVQVTCGTT TLNGLWLDDT VYCPRHVICT
AEDMLNPNYE DLLIRKSNHS FLVQAGNVQL RVIGHSMQNC
LLRLKVDTSN PKTPKYKFVR IQPGQTFSV LACYNGSPSG
VYQCAMRPNH TIKGSFLNGS CGSVGFNIDY DCVSFCYMHH
MELPTGVHAG TDLEGKFYGP FVDRQTAQAA GTDTTITLNV
LAWLYAAVIN GDRWFLNRFT TTLNDFNLVA MKYNYEPLTQ
DHVDILGPLS AQTGIAVLDM CAALKELLQN GMNGRTILGS
TILEDEFTPF DVVRQCSGVT FQGKFKK

SARS-COV-2 3CLpro is conserved, and shares 99.02% sequence identity with SARS-COV 3CLpro. Mutations are thought to disrupt important hydrogen bonds and alter the receptor binding site of SARS-COV-2 3CLpro (id).

The following table shows the sequence homology of protease inhibitors among a variety of different viruses:

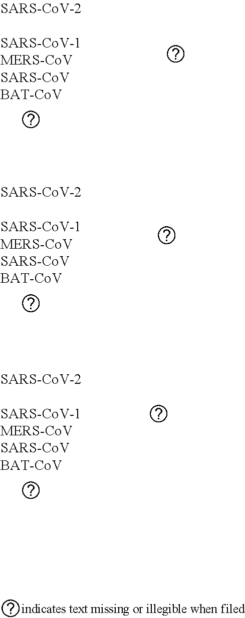

The Protein Data Bank (RCSB PDB) is replete with structures of SARS-COV-2 wild-type (WT) 3CLpro. 3CLpro WT homodimers are a 67.60 kDa, heart-shaped complex. Each 3CLpro chain consists of three domains. Domain I (aa. 8-101) and Domain II (aa. 102-184) have a predominantly β-sheet structure, form the active site, and contribute to dimerization. Domain III (aa. 201-303) is substantially a-helical and is the primary determinant of dimerization. The active site of WT 3CLpro contains a catalytic dyad of His41 and Cys145, and an oxyanion hole formed by the main chain amide groups of Glyl43 and Cys145. During catalysis, His41 deprotonates the γ-thiol group of Cys145 to generate a nucleophile. Nucleophilic attack at the main chain carbonyl carbon of the P1 residue (immediately preceding the substrate scissile bond) forms a tetrahedral oxyanion intermediate. Heterolytic fission of the scissile bond stabilizes this intermediate, forms the acyl-enzyme intermediate, and releases the peptide downstream of the scissile bond. Finally, the active site is regenerated as a catalytic water molecule deacylates the acyl-enzyme intermediatc.

Functionally, 3CLpro recognizes a hydrophobic substrate residue at P2 (usually Phe or Leu), a Gln at P1, and Ser, Val, Asn, or Ala residues at P1′. This recognition motif is found in multiple sites of the viral polyproteins, which arc cleaved by 3CLpro to form mature nsp5-16. This consensus sequence is also present in proteins of the host innate immune pathway and therefore 3CLpro may blunt the immediate antiviral immune response via proteolysis.

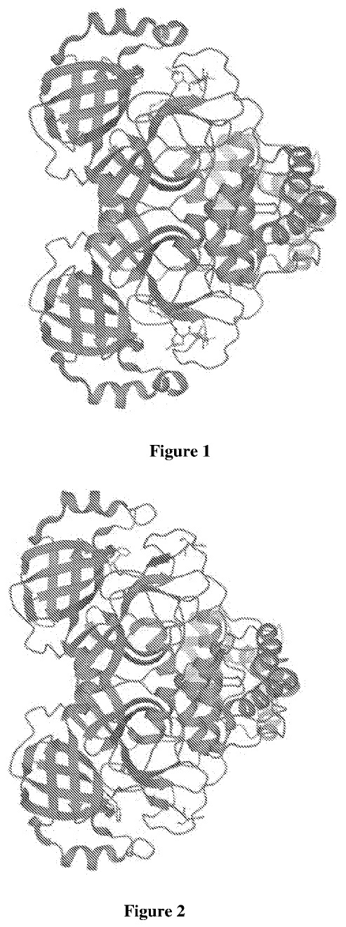

Pfizer has released the crystal structure of 3CLpro complexed with its protease inhibitor, PF07321332 (PDB: 7RFS), disclosed in Greasley et al., “Structure of SARS-COV-2 main protease in complex with a covalent inhibitor,” (2021) Science 374:1586-1593, and shown in FIG. 1.

There are also six crystal structures of 3CLpro with distinct substrate peptides, along with the corresponding Protein Data Bank (PDB) accession numbers. The cleavage sites are shown in the table below:

### 3CLpro Cleavage Sites

| P₄-P₁ | —— | P₁′-P₄′ |
|---|---|---|
| nsp4 . . . AVLQ | \| | SGFR . . . nsp5 |
| nsp5 . . . VTFQ | \| | SAVK . . . nsp6 |
| nsp6 . . . ATVQ | \| | SKMS . . . nsp7 |
| nsp7 . . . ATLQ | \| | AIAS . . . nsp8 |
| nsp8 . . . VKLQ | \| | NNEL . . . nsp9 |
| nsp9 . . . VRLQ | \| | AGNA . . . nsp10 |
| nsp10 . . . PMLQ | \| | SADA . . . nsp11 |
| nsp12 . . . TVLQ | \| | AVGA . . . nsp13 |
| nsp13 . . . ATLQ | \| | AENN . . . nsp14 |
| nsp14 . . . TRLQ | [symbol] | SLEN . . . nsp15 |

The crystal structures are identified below, and shown in FIGS. 2-7:

nsp4-nsp5 (PDB: 7N89), a room-temperature X-ray structure of SARS-COV-2 main protease C145A mutant in complex with substrate Ac-SAVLQSGF-CONH2, which is published at Kneller et al., “Michaelis-like complex of SARS-COV-2 main protease visualized by room-temperature X-ray crystallography,” (2021) IUCrJ 8:973-979, and shown in FIG. 2.

nsp6-nsp7 (PDB: 7DVX), a SARS-COV-2 Mpro mutant (H41A) in complex with nsp6|7 peptidyl substrate, published at DOI: 10.2210/pdb7DVX/pdb, and shown in FIG. 3.

nsp8-nsp9 (PDB: 7MGR), the SARS-COV-2 main protease in complex with N-terminal autoprocessing substrate, published in MacDonald, et al., “Recognition of Divergent Viral Substrates by the SARS-COV-2 Main Protease,” (2021) ACS Infect Dis 7:2591-2595, DOI: 10.2210/pdb7MGR/pdb, and shown in FIG. 4.

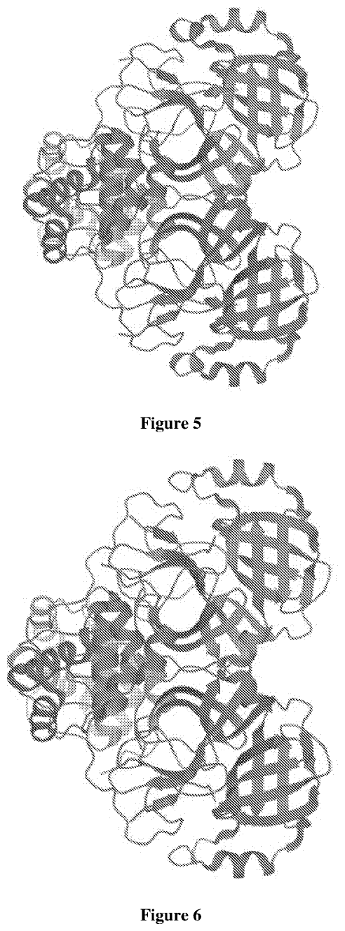

nsp9-nsp10 (PDB: 7DVY), a SARS-COV-2 Mpro mutant (H41A) in complex with the nsp9|10 peptidyl substrate, disclosed at DOI: 10.2210/pdb7DVY/pdb, and shown in FIG. 5.

nsp14-nsp15 (PDB: 7DW6), a SARS-COV-2 Mpro mutant (H41A) in complex with nsp14|15 peptidyl substrate, disclosed at DOI: 10.2210/pdb7DW6/pdb, and shown in FIG. 6.

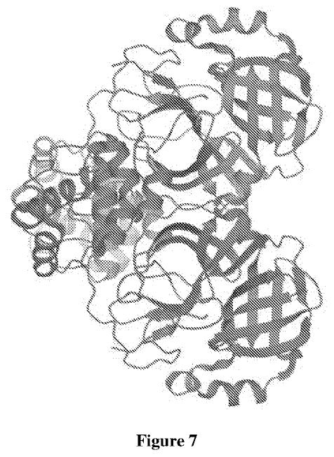

nsp15-nsp16 (PDB: 7DW0), a SARS-COV-2 Mpro mutant (H41A) in complex with nsp15|16 peptidyl substrate, disclosed at DOI: 10.2210/pdb7DW0/pdb, and shown in FIG. 7.

### In Silico Model

### Methods:

### Residue Scanning+MMGBSA

In one embodiment, the predictive model described herein uses a combination of two computational methods, residue scanning and MMGBSA from Schrödinger to predict the resistance mutations.

### Step-1 Residue Scanning

In the first step, the predictive model uses residue scanning of all the active site residues identified based on the crystal structure of the Pfizer molecule complexed with SARS-COV-2 3CLpro. In this step, it mutates the residue in to all 19 possible residues and calculates the binding affinity change (ΔΔG). The calculation is based on physics-based scoring function implemented by Schrödinger. Here we did not apply any residue flexibility. The main purpose of this step to remove the mutations which do not affect the binding of pfizer drug molecule which means ΔΔG =0 or ΔΔG<0. Mutations which do not affect the binding of drug molecule is likely to change the conformation when the sidechain of backbone flexibility applied because they are in energy minimum state so this is the central hypothesis to perform this step first before applying 2^{nd} step of MMGBSA with sidechain flexibility. Residue scanning is faster than MMGBSA so we can remove the mutations quickly which do not affect the binding of drug and rest of the mutations which decrease the binding of drug are being used for 2^{nd} step of MMGBSA calculations with side chain flexibility. By incorporating side chain flexibility this mutations might change from decreasing binding affinity (ΔΔG>0) to maintain (ΔΔG=0) or increase (ΔΔG<0) the binding affinity

Residue scanning is also performed on all 6 crystal structures of 3CLpro with 6 distinct substrates.

### Protein-Protein Complexes

Residue scanning module in Schrodinger was used and explored for protein-protein complexes, it was first time we have explored for protein and small drug molecule.

Mutates the residue and calculates ΔΔG (kcal/mol)

Explore the conformational space of 3 residues

Residue scanning explores the conformation of mutated residue and the one residue from both side of mutated residue. So total 3 residue conformational space is explored by residue scanning.

$$Δ Δ {G}_{( bind )} = Δ {G}_{( mutant )} - Δ {G}_{( wild type )}$$

### ΔΔG>0, Decreased Binding

### ΔΔG<0, Increased Binding

This is a thermodynamic cycle concept where (ΔΔG>0) means decreased binding and (ΔΔG<0) means increased binding

Filtered mutations:→ΔΔG>0 retained

Mutations for the 2^{nd} step, which show ΔΔG>0 in the residue scanning step, are considered mutations of interest.

### Step-2 MMGBSA Calculations

The calculations are rigorous and computationally expensive because of the flexibility of residues. Sidechain flexibility of residues can be implemented in this approach, so it is computationally a little bit more expensive then residue scanning.

Calculate ΔG (kcal/mol) for wild type and mutations

This MMGBSA step provides the binding free energy change (ΔG) due to binding, not ΔΔG (Which is binding free energy change due to mutations). So, ΔG was calculated for all selected mutations from step 1, then ΔG was calculated for the wild type protease, then the difference between the ΔG of the mutation and the ΔG of the wild type was taken to arrive at the ΔΔG.

Residues around 8 Å of molecule—This relates to the incorporation of sidechain flexibility in MMGBSA calculations. The residues were kept 8 Å around the drug molecule within the binding pocket are flexible means the sidechains of residues which are 8 Å around the drug are kept flexible. This flexibility was included in MMGBSA ΔG calculations.

Step-3 Mutations with ΔΔG>0 for drug and ΔΔG<=0 for substrate taken for Single nucleotide substitution (SNS) check and mutated residues associated with SNS were considered as drug resistance mutations

The GISAID Initiative promotes the rapid sharing of data from all influenza viruses and the coronavirus causing COVID-19. This initiative has identified four mutations in the active sites from different countries and strains (https://www.gisaid.org). These mutations include:
Y54C—Reported in March 2020 in Malaysia. Not many reports available regarding occurrence of this mutation. (predicted resistance mutation)
N142S—Reported 17 times in 5 different countries
T190I—Occurred 110 times (0.03% of the sequenced NSP5) from 15 different countries. (predicted resistance mutation)
A191V-March 2020 (South Africa), January 2021 (USA). Occurrence rate 0.30% of the total sampled sequence in 34 countries.

It is predicted that these variants, which occurred randomly and without exposure to the Pfizer protease inhibitor, would likely be resistant to the Pfizer protease inhibitor.

### Example 2: Evaluation of the Model Using HBV and HIV

### Introduction

The discovery of effective antiviral drugs revolutionized world health saving millions of lives. Despite these medical advances, selection of resistant strains is a persistent problem leading to viral break-through and mitigating efficacy [1-4]. There are several mechanisms reported for the development of drug-resistance [5,6]. The standing out random mutations in viral genes which alter the binding of drug with its corresponding protein target is the main mechanism of acquiring drug resistance in viruses [6]. The mutation rate in viruses is very high, for RNA viruses, it is estimated 10-4 per nucleotide per replication while in DNA viruses it is 10-8 per nucleotide per replication [7,8]. Thus, the drug resistance is one of the greatest risks to the public's health and a priority across the globe. Hence, a prior knowledge of drug resistance against drug targets and resistance mechanism is of great importance to develop more effective and long-lasting drug treatments.

Resistant virus is typically selected by maintaining an infected in vitro culture under drug pressure for months, sometimes years, with no guarantee that resistance emerges in cellular conditions [9]. In certain situations, resistance appears exclusively in clinical settings requiring hasty characterization of the mutation and viral species during trials [10].

The capability to predict resistance expedites understanding of antiviral efficacy, anticipates activity against existing mutant strains, delivers mechanistic insight into how certain mutants confer resistance, forecasts species that may develop in clinical settings, and provides broad utility and benefit to infectious disease drug discovery [11].

Numerous efforts have been made to study the drug resistance mechanism induced by mutations and to develop the tools to predict the drug resistance mutations. One group of prediction models include sequence-based approaches which use various machine learning methods which primarily rely on primary sequences of the protein or genotypic sequence data Their prediction accuracies are dependent on availability of large and diverse training set [12-15]. The main advantage of these methods is computationally efficient, but they cannot predict the drug resistant mutations for novel drug molecules as they lack training set data. Without 3-D structural information and enzymatic function of the mutated residues, this group of models fail to capture the bridges between genetic viral mutations and structural changes due to corresponding phenotypic mutations [11,16,17]. Another group of the prediction methods is based on the 3-D structure of the target proteins. From last few decades, the availability of large number of 3-D structures of the protein targets enable to implement various structure-based molecular modeling approaches to study the binding interactions and binding free energies of drug molecules with their corresponding protein targets. The binding free energies are crucial to facilitate the prediction of drug resistance mutations [18-21]. Although these methods are advantageous over sequence-based methods, they can be time-consuming or produce low predictive accuracy. Hence, there is a need for novel structure-based methods of optimum balance between computational efficiency and predictive accuracy [22].

Molecular Mechanics-Generalized Born Surface Area (MM-GBSA) is one of the widely used structure-based approaches to predict the binding affinities in protein-ligand or protein-protein complexes [23-26]. Recently Schrodinger has evaluated physics-based scoring function with MM-GBSA model, Residue Scanning to calculate binding free energies of protein-protein complexes due to single point mutations [23, 24]. Moreover, they showed that MM-GBSA has slightly better accuracy compared to other prediction methods, PoPMuSiCsyn FoldX and Rossetta in predicting binding affinities due to single point mutations in protein-protein complexes [23,24]. Although promising, MM-GBSA has not been explored for small molecules with their protein targets to calculate binding affinity due to single amino acid mutations.

Typically, resistance mutations in viruses meet three requirements: 1) the mutation decreases binding of inhibitor, 2) the mutation retains affinity for native substrate and maintains essential function, and 3) the mutant residue is accessible by a single nucleotide substitution (SNS) in the wild-type codon [16, 30]. Only few active-site mutations meet all three criteria to deliver resistance, and this hypothesis enable the computational prediction of selective escape mutations using structure-based methods. Here, we predicted the binding affinities by MM-GBSA calculations with the help of two Schrödinger suite modules, Residue scanning and Prime MM-GBSA in context to predict drug resistance. We first implemented and assessed this approach to predict the resistance mutations in HIV reverse transcriptase (RT) for (-) FTC and later predicted the resistance mutations in HBV capsid for GLP-26 and in SARS-COV-2 3CLpro for PF-07321332. To validate the approach, we selected 5 mutations on HBV capsid from the predictions and performed the mutagenesis experiments. Our approach not only identified the clinical resistance mutations in HIV RT for (-) FTC but accurately predicted GLP-26 resistance mutations in HBV capsid. Although the approach has optimum balance between efficiency and accuracy, there is room to improve predicting binding affinities accurately to compare with experimental values.

### Results and Discussion

The purpose of this study to assess and predict the drug resistance mutations using the computational workflow of MM-GBSA calculations with Residue Scanning and Prime MM-GBSA calculation to achieve balance between better predictive accuracy and computational efficiency. We tested this approach on three protein-drug complexes, HIV RT complexed with (-) FTC, HBV capsid with GLP-26 and SARS-COV-2 3CLpro with PF-07321332. The pharmacological activities and resistance mutations of (-) FTC have been studied rigorously by our group (Ref). Moreover, the clinically significant resistance mutations are reported and well-studied [31]. Thus, HIV RT with (-)-FTC was the ideal system to begin with and to test our computational protocol to predict the resistance mutations using our computational protocol.

The approach begins with Residue Scanning followed by Prime MM-GBSA calculations, as described above in Example 1.

Residuc Scanning generates the mutations for specified residues using Prime rotamer search algorithm then performs MM-GBSA refinement of the bound and unbound state for each system for both wild type and mutant protein structures. It is a non-rigorous, computationally efficient method. The protein backbone was kept, and the neighboring side chains were fixed; thus, this approach quickly screens the mutations and predicts the binding affinities. The main aim of implementing this approach is to filter out the mutations at the beginning which show the increase in predicted binding affinities (ΔΔG<0) of drug/substrate molecules with their proteins. Mutations associated with a decrease in binding affinities (ΔΔG>0) can be further explored to calculate binding affinities with side-chain flexibility in the binding sites using molecular modeling software, such as Prime MM-GBSA.

It was hypothesized that mutations which increase the binding affinities (ΔΔG<0) are in energy minimum conformations [23], and so by providing side-chain flexibility, it is less likely to change binding affinities from an increase (ΔΔG<0) to a decrease (ΔΔG>0) in binding affinities. Moreover, the mutations with ΔΔG>0 for the drug complexes and ΔΔG<=0 for the native substrate complexes are of interest, as they are mutations a virus could select as potential resistant mutations. That is, the mutations would cause the drug to not bind in the binding pocket of the enzyme, while not significantly affecting the binding of the enzyme to the substrate.

### Materials and Methods

### 1.1 Test System Selection and Preparation

HIV is known to produce mutations when exposed to (-)-FTC. To test the effectiveness of the predictive model described herein, a crystal structure of HIV RT complexed with (-) FTC (PDB ID-6UJX) was selected, and assessed to predict resistance mutations. The crystal structure of GLP-26 with the HBV capsid has not been resolved yet, so a previously published modeled complex of GLP-26 with HBV capsid was selected for this study [32].

As discussed above in Example 1, an additional system, SARS-COV-2 3CLpro for PF-07321332 (PDB ID-7RFS) was used to predict the resistance mutations. To calculate free energy change in HIV RT with natural substrate, HIV RT complexed with dCTP (PDB ID-6UIT) while in case of HBV capsid, GLP-26 binds between two dimeric subunits thus HBV capsid tetramer (PDB ID-1QGT) were taken.

Because of no 3D structure of SARS-COV-2 3CLpro complexed with substrate peptide, covalent docking of the substrate peptides was performed. The cysteine 145 residue which is available in the binding site of SARS-COV-2 3CLpro is a reactive residue that can form a covalent bond with the substrate or drug molecules. The substrates are peptides and so carbonyl groups are the reactive functional groups for forming the covalent bond with Cys145 thus the nucleophilic addition to double bond mechanism was selected in covalent docking. Finally, the docked poses of the covalently linked substrates are to be visualized and rank ordered by energy and the docked score.

The PDB structures and modeled structures were prepared using Protein Preparation Wizard in Maestro (Schrödinger Release 2020-4; Schrödinger) Missing residues and loops were added and minimized using Prime [33,34]. Crystallographic waters were deleted, and the hydrogen bonding network was optimized using Epik at neutral pH [35]. The final structures were minimized with heavy atom restraints using the OPLS3e force field. The minimization was terminated when the heavy-atom root mean square deviation reached 0.3 Å.

### 1.2 Residue Scanning

The binding site residues of drug/substrate is defined by Binding Site object in Maestro (Schrödinger Release 2020-4; Schrödinger) The change in binding affinities of the residues due to mutations were calculated using Residue Scanning module in BioLuminate (Schrödinger Release 2020-4; Schrödinger). In the residue scanning panel, all (allowed) mutations for the interest of residue and side-chain prediction with backbone minimized option with cutoff 0.0 Å were selected for the refinement of the mutated residue. The residue scanning use MM-GBSA refinement without any sidechain and backbone flexibility.

### 1.3 Primc MM-GBSA Calculations

The WT-drug/substrate and MUT-drug/substrate complexes which show greater than 0 kcal/mol binding affinity change in Residue Scanning calculations are selected for Prime MM-GBSA estimation with sidechain flexibility. For the covalent systems, the covalent bond was removed for MM-GBSA calculations. VSGB (variable-dielectric generalized Born) solvation model and OPLS3e force field were utilized during MM-GBSA calculations. Side-chain flexibility was incorporated to the residues within 8 Å of the drug molecule. The change in binding affinities, ΔGMUT and ΔGWT were calculated separately by Prime MMGBSA and difference between them was considered to calculate change in the binding affinity due to mutations,

$$Δ Δ {G}_{bind} = Δ {G}^{MUT} - Δ {G}^{WT} \qquad ( 1 )$$

### 1.4 Cell Lines

Wild type HBV DNA was amplified and cloned as previously described (38, 39). Five HBV core mutants (F23Y, L30F, T33Q, I105F and T109I) were created by substituting nucleotides to change the codon as indicated below using the Quik-Change II Site-Directed Mutagenesis Kit (Agilent, Santa Clara, CA, USA). Primers used for site directed PCR mutagenesis are described in the following table.

| Pair name | Sequence Information |
|---|---|
| core T109I | 5′-ctataactgtttctcttccaaaaatgagacaaga |
| mutant c326t | aatgtgaaaccac-3′ |
| | 5′-tgtgtttcacatttcttgtctcatttttggaaga |
| | gaaacagttatag-3′ |
| core I105F | 5′-ccaaaagtgagacaagaaaagtgaaaccacaaga |
| mutant a313t | gttgc-3′ |
| | 5′-gcaactcttgtggtttcacttttcttgtctcact |
| | ttgg-3′ |
| core T33Q | 5′-atacagagctgaggcctgatctagaagatctcgt |
| mutant a97c_ | actgaaggaaaga-3′ |
| c98a_c99g | |
| | 5′-tctttccttcagtacgagatcttctagatcaggc |
| | ctcagctctgtat-3′ |
| core L30F | 5′-gcggtatctagaaaatctcgtactgaaggaaaga |
| mutant c88t | agtc-3′ |
| | 5′-gacttctttccttcagtacgagattttctagata |
| | ccgc-3′ |
| core F23Y | 5′-tctcgtactgaaggaaagtagtcagaaggcaaaa |
| mutant t68a | acg-3′ |
| | 5′-cgtttttgccttctgactactttccttcagtacg |
| | aga-3′ |

The core gene of the mutants were sequenced bidirectionally by GENEWIX (New Jersey, USA) to confirm the introduction of mutations.

### 1.5 Compound Synthesis

GLP-26 and GSL4 were prepared in-house according to published procedures [36, 37]. Both compounds had a purity of >95% as determined by 1H, 13C, 19F nuclear magnetic resonance (NMR) and high-pressure liquid chromatography (HPLC) analysis. Entecavir (ETV) was purchased from commercial vendors and confirmed at >95% purity using standard analytical methods such as mass spectrometry and NMR.

### 1.6 Cell Lines

HepNTCP-DL cells were maintained in Dulbecco's modified minimal essential medium (DMEM) supplemented with 10% FBS and 0.1 mM non-essential amino acids (NEAA).

1.7 Transfection of Full Length HBV DNA into HepNTCP-DL Cells

Full length HBV DNA wild-type and core mutants were prepared for transfection as previously described [38]. HepNTCP-DL cells were seeded in either 96 or 24 well collagen-coated plates in DMEM supplemented with 10% FBS and 0.1 mM NEAA and maintained in a tissue culture incubator at 37° C. with 5% CO2. The cells were 90% confluent the next day and medium was changed to medium DMEM supplemented with 3% FBS and 0.1 mM NEAA. Transfection of HBV DNA was performed with Lipofectamine 3000 reagent (Invitrogen, Carlsbad, CA, United States) according to the manufacturer's instructions. Twenty-four hours after transfection, the medium was changed and reincubated with drug-free medium or medium containing several different concentrations of either GLP-26 or GSL4. Medium and cells (rinsed 3 times with ice cold PBS) were harvested 3 days later. The efficiency of transfection was monitored by cotransfecting a B-galactosidase expression plasmid, pCMVB (CLONTECH Laboratories Inc., Palo Alto, California, USA). Assays for β-galactosidase in extracts of HuH-7 cells were performed as described [39]. Ex-periments were performed in triplicate.

### 1.8 Analysis of HBV HBsAg and HBeAg Production

Levels of HBsAg and HBeAg secreted in the culture medium were measured by using an HBsAg or HBeAg enzyme-linked immunosorbent assay (ELISA) kit (Bi-oChain Institute Inc. Hayward, CA) respectively, according to the manufacturer's protocol. The concentration of compound that reduced levels of secreted HBsAg or HBeAg by 50% (EC₅₀) was determined by linear regression.

1.9 Anti-HBV Activity Using qPCR Assay for the Detection of HBV DNA

DNA was extracted and purified from HepNTCP-DL HBV DNA transfected cells using a commercially available kit (plasmid miniprep kit; Qiagen). The in vitro anti-HBV activity of the synthesized compounds were assessed by real-type PCR (qPCR) as previously described [40]. The concentration of compound that inhibited HBV DNA replication by 50% (EC₅₀) was determined by linear regression.

The data show that the predictive model identified potential mutations when HIV is exposed to (-)-FTC, and these mutations correlated with mutations actually observed when (-)-FTC has been administered to HIV positive patients. This shows that the predictive model can accurately predict mutations of potential interest when a virus is exposed to a particular drug, subject to the caveat that the method requires the crystal structures of the drug and the target viral enzyme, as well as crystal structures of the enzyme with one or more the substrates to which it binds.

### Example 3: Application of the Model to HIV-Reverse Transcriptase Using-(−)-FTC-TP

Using the model described herein, mutations in HIV-Reverse Transcriptase that would appear following exposure to-(−)-FTC-TP were evaluated. There are known mutations associated with this particular active agent, so these known mutations were compared with predicted mutations to show the strength of the predictive model.

One such known mutation is M184V. It is described in the following two papers:

PDB ID: 6UJX-HIV RT in complexed with (-)-FTC-TP, HIV-1 wild-type reverse transcriptase-DNA complex with (-)-FTC-TP, disclosed in Hung et al., “Elucidating molecular interactions of L-nucleotides with HIV-1 reverse transcriptase and mechanism of M184V-caused drug resistance,” (2019) Commun Biol 2:469-469

PDB ID: 6UIT-HIV RT in complexed with dCTP, disclosed in Hung et al., “Elucidating molecular interactions of L-nucleotides with HIV-1 reverse transcriptase and mechanism of M184V-caused drug resistance,” (2019) Commun Biol 2:469-469

Using the predictive model described herein, the following mutations were predicted when -(-)-FTC-TP is used to treat HIV by inhibiting the HIV-Reverse Transcriptase.

The model predicted two most important clinical mutations as per below table where those mutations shown in bold color are reported drug resistance mutations in the clinic.

| WT | Predicted Resistant Mutations |
|---|---|
| M184 | I, V, A, R, N, D, C, Q, G, L, K, P, S, T, W, Y |
| I63 | D, E |
| K65 | R |
| K66 | I, P, T, G |
| D113 | E |
| Y115 | A, R, N, D, C, Q, E, G, H, I, L, K, P, S, T, V |
| Q151 | E, I, C, D, P |
| Q152 | D, E |
| Y183 | D, E |

Thus, the predictive method is able to identify known mutations that occurred in the wild-type reverse transcriptasc following administration of-(-) FTC-TP.

### Predicted Drug Resistance Mutations in HBV Capsid for Drug Molecule GLP-26

GLP-26 binding between two monomers of HBV capsid and when there is no drug interacts with monomer then monomers interact with each other. So, GLP-26 disrupts the binding of two monomer. Here there is no substrate or endogenous ligand to study the mutations so here we studied monomer binding with another monomer means protein-protein interactions between two monomer which is considered as unbound form in below chart.

The list of the mutations are below in tabular form. The B and C mentioned in the bracket are the monomers B and monomer C. Bold color shows mutations affect the binding of GLP-26 at higher degree means this mutations could reduce the binding affinity at high degree.

| | PREDICTED | | PREDICTED |
|---|---|---|---|
| | RESISTANCE | | RESISTANCE |
| WT | MUTATIONS | WT | MUTATIONS |
| (B) S106 | W, Y, F, L, C | (B)F23 | M, L |
| (B)L140 | P, C, I, V, H, M, Y | (B)I105 | M |
| (C) T128 | K, R, P, I, M | (B)P138 | H |
| (B) L30 | W, H | (C) P129 | H |
| (B) W102 | G, C, L, Y, F, S | (B) F122 | V |
| (B) Y118 | Q, D, C, W, E, H, K, | (C) R133 | T |
| | R, F | | |
| (C) P134 | L, H, S | | |
| (C) R133 | I, C, L | | |
| (C) W125 | G | | |
| (B) T33 | R, M | | |
| (B) F110 | I | | |
| (B) P25 | L, Q, R | | |

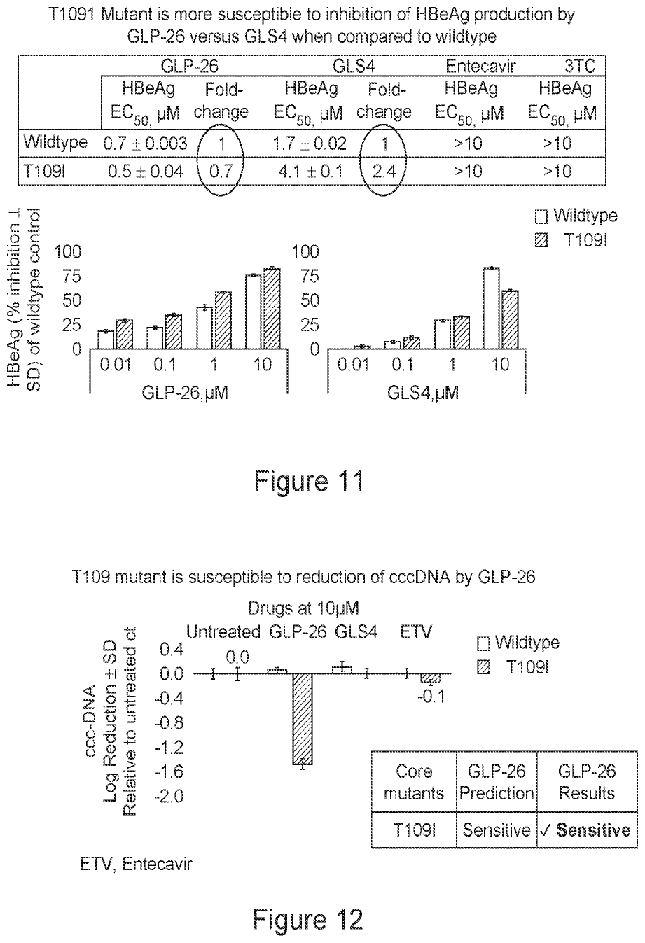

The T190I mutation is naturally occurring, and most HBV capsid modulating drugs arc resistant to this mutation, but the predictive model described herein did not predict this mutation as a resistant mutation for GLP-26. The experimental assay was performed as mentioned above for HBV to study the mutation T190I, and this mutation was not found to be a drug resistant mutation as predicted. This is shown in FIGS. 11 and 12, which are charts showing that the T109I mutant is more susceptible to inhibition of HBeAg production by GLP-26 versus GLS4 when compared to wildtype (FIG. 11) and is susceptible to reduction of cccDNA by GLP-26 (FIG. 12).

### Example 4: Compound-1 SARS COV-2 3CLpro Resistance Compound

### Background

The COVID-19 pandemic caused by the novel coronavirus SARS-COV-2 in late 2019 continues to pose a threat to the world population as the virus constantly mutates.

Coronavirus main protease (3CLpro) is a cysteine protease, responsible for processing viral polyproteins to produce non-structural proteins, which plays an important role in the replication cycle and therefore is considered a prominent target for antiviral drugs.

Pfizer's newly FDA-approved nirmatrelvir, offers hope on the therapeutic front in certain populations.

RNA viruses have inherently high mutation rates, which can easily escape selection pressure through mutation of vital target amino acid residues.

Due to challenges in viral selection, it remains to be seen what mutant will be selected culture and in humans that confers resistance to antivirals.

Goal: Implement well-developed computational approach for predicting resistance mutations in SARS-COV-2 3CLpro for nirmatrelvir and in-house inhibitor Compound-1 based on hypothesis that resistant mutations will decrease inhibitor binding while retaining binding affinity of native substrate peptides.

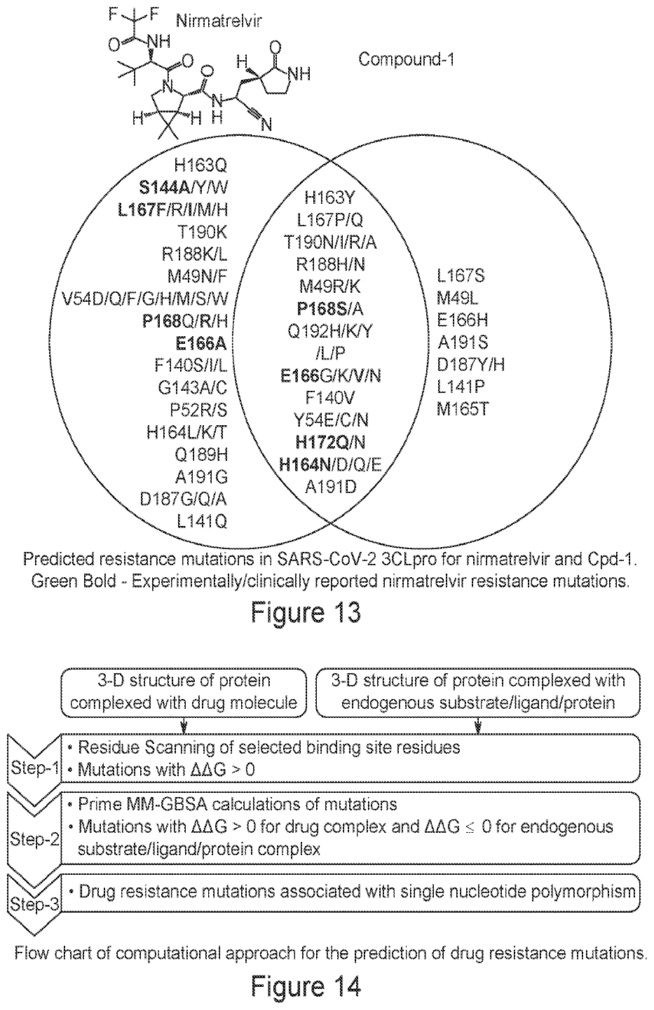

Predicted resistance mutations in SARS-COV2 3CLpro for nirmatrelvir and Compound 1 are shown in FIG. 13, and those for Compound 1 are summarized in the following table.

### Predicted Resistance Mutations in SARS-COV-2 3CLpro for Compound 1

| | PREDICTED | | PREDICTED |
|---|---|---|---|
| | RESISTANT | | RESISTANT |
| WT | MUTATIONS | WT | MUTATIONS |
| D187 | Y, H | R188 | H, N |
| P168 | S, A | M165 | T |
| Q192 | K, H, Y, P, L | H163 | Y |
| E166 | V, H, N, K, G | Y54 | E, C, N |
| L167 | P, Q, S | H164 | D, N, Q, E |
| M49 | K, L, R | H172 | Q, N |
| L141 | P | A191 | D, S |
| T190 | A, R, I, N | F140 | V |

Bold—predicted Cpd-1 resistance mutations with ΔΔG>3 kcal/mol

Nirmatrelvir resistance mutations in SARS-COV-2 3CLpro were predicted using the methods described herein. The computational approach has successfully recaptured the experimental/clinical resistance mutations for nirmatrelvir. From the predicted resistance mutations, Y54C and T109I mutants were reported as natural variants. Nirmatrelvir showed 1.6-fold change in IC₅₀ against Y54C 3CLpro mutant which validated the predictions. Inhibitory activity of Cpd-1 against mutant Y54C 3CLpro is ongoing.

Cpd-1 is predicted to have better resistance profile compared to nirmatrelvir.

### References

1. Perrin, L.; Telenti, A. HIV treatment failure: Testing for HIV resistance in clinical practice. Science 1998, 280, 1871-1873, doi: 10.1126/science.280.5371.1871.
2. Elhence, A.; Singh, A.; Anand, A.; Kumar, R.; Ashraf, A.; Kumar, S.; Pradhan, D.; Pathak, P.; Vaishnav, M.; Rajput, M. S.; et al. Real-world re-treatment outcomes of direct-acting antiviral therapy failure in patients with chronic hepatitis c. J Med Virol 2021, 93, 4982-4991, doi: 10.1002/jmv.26971.
3. Stuyver, L. J.; Locarnini, S. A.; Lok, A.; Richman, D. D.; Carman, W. F.; Dienstag, J. L.; Schinazi, R. F. Nomenclature for antiviral-resistant human hepatitis b virus mutations in the polymerase region. Hepatology 2001, 33, 751-757, doi: 10.1053/jhep.2001.22166.
4. Burrel, S.; Topalis, D.; Boutolleau, D. [herpes simplex virus resistance to antivirals]. Virologie (Montrouge) 2020, 24, 325-342, doi: 10.1684/vir.2020.0864.
5. Borst, P. Genetic mechanisms of drug resistance. A review. Acta Oncol 1991, 30, 87-105, doi: 10.3109/02841869109091819.
6. Erickson, J. W.; Burt, S. K. Structural mechanisms of HIV drug resistance. Annu Rev Pharmacol Toxicol 1996, 36, 545-571, doi: 10.1146/annurev.pa.36.040196.002553.
7. Mason, S.; Devincenzo, J. P.; Toovey, S.; Wu, J. Z.; Whitley, R. J. Comparison of antiviral resistance across acute and chronic viral infections. Antiviral Res 2018, 158, 103-112, doi: 10.1016/j.antiviral.2018.07.020.
8. Vere Hodge, A.; Field, H. J. General mechanisms of antiviral resistance. Genetics and Evolution of Infectious Disease 2011, 339-362, doi: 10.1016/B978-O-12-384890-1.00013-3.
9. Vacca, J. P.; Dorsey, B. D.; Schleif, W. A.; Levin, R. B.; McDaniel, S. L.; Darke, P. L.; Zugay, J.; Quintero, J. C.; Blahy, O. M.; Roth, E.; et al. L-735,524: An orally bioavailable human immunodeficiency virus type 1 protease inhibitor. Proc Natl Acad Sci USA 1994, 91, 4096-4100, doi: 10.1073/pnas.91.9.4096.
10. Larder, B. A.; Kemp, S. D. Multiple mutations in HIV-1 reverse transcriptase confer high-level resistance to zidovudinc (azt). Science 1989, 246, 1155-1158, doi: 10.1126/science.2479983.
11. Cao, Z. W.; Han, L. Y.; Zheng, C. J.; Ji, Z. L.; Chen, X.; Lin, H. H.; Chen, Y. Z. Computer prediction of drug resistance mutations in proteins. Drug Discov Today 2005, 10, 521-529, doi: 10.1016/S1359-6446 (05) 03377-5.
12. Steiner, M. C.; Gibson, K. M.; Crandall, K. A. Drug resistance prediction using deep learning techniques on HIV-1 sequence data. Viruses 2020, 12, 560, doi: 10.3390/v12050560.
13. Beerenwinkel, N.; Däumer, M.; Oette, M.; Korn, K.; Hoffmann, D.; Kaiser, R.; Lengauer, T.; Selbig, J.; Walter, H. Geno2pheno: Estimating phenotypic drug resistance from HIV-1 genotypes. Nucleic Acids Research 2003, 31, 3850-3855, doi: 10.1093/nar/gkg575.
14. Riemenschneider, M.; Hummel, T.; Heider, D. Shiva-a web application for drug resistance and tropism testing in HIV. BMC Bioinformatics 2016, 17, 314, doi: 10.1186/s12859-016-1179-2.
15. Bonet, I. Machine learning for prediction of HIV drug resistance: A review. Current Bioinformatics 2015, 10, 579-585.
16. Khalid, Z.; Sezerman, O. U. Prediction of HIV drug resistance by combining sequence and structural properties. IEEE/ACM Trans Comput Biol Bioinform 2018, 15, 966-973, doi: 10.1109/TCBB.2016.2638821.
17. Weber, I. T.; Harrison, R. W. Tackling the problem of HIV drug resistance. Postepy Biochem 2016, 62, 273-279.
18. Jenwitheesuk, E.; Samudrala, R. Prediction of HIV-1 protease inhibitor resistance using a protein-inhibitor flexible docking approach. Antivir Ther 2005, 10, 157-166.
19. Toor, J. S.; Sharma, A.; Kumar, R.; Gupta, P.; Garg, P.; Arora, S. K. Prediction of drug-resistance in HIV-1 subtype c based on protease sequences from art naive and first-line treatment failures in north India using genotypic and docking analysis. Antiviral Res 2011, 92, 213-218, doi: 10.1016/j.antiviral.2011.08.005.
20. Hou, T.; Yu, R. Molecular dynamics and free energy studies on the wild-type and double mutant HIV-1 protease complexed with amprenavir and two amprenavir-related inhibitors: Mechanism for binding and drug resistance. J Med Chem 2007, 50, 1177-1188, doi: 10.1021/jm0609162.
21. Agniswamy, J.; Louis, J. M.; Roche, J.; Harrison, R. W.; Weber, I. T. Structural studies of a rationally selected multi-drug resistant HIV-1 protease reveal synergistic effect of distal mutations on flap dynamics. PLOS ONE 2016, 11, e0168616, doi: 10.1371/journal.pone.0168616.
22. Hao, G. F.; Yang, G. F.; Zhan, C. G. Structure-based methods for predicting target mutation-induced drug resistance and rational drug design to overcome the problem. Drug Discov Today 2012, 17, 1121-1126, doi: 10.1016/j.drudis.2012.06.018.
23. Beard, H.; Cholleti, A.; Pearlman, D.; Sherman, W.; Loving, K. A. Applying physics-based scoring to calculate free energies of binding for single amino acid mutations in protein-protein complexes. PLOS One 2013, 8, e82849, doi: 10.1371/journal.pone.0082849.
24. Duan, J.; Lupyan, D.; Wang, L. Improving the accuracy of protein thermostability predictions for single point mutations. Biophys J 2020, 119, 115-127, doi: 10.1016/j.bpj.2020.05.020.
25. Genheden, S.; Ryde, U. The mm/pbsa and mm/gbsa methods to estimate ligand-binding affinities. Expert Opin Drug Discov 2015, 10, 449-461, doi: 10.1517/17460441.2015.1032936.
26. Wang, E.; Sun, H.; Wang, J.; Wang, Z.; Liu, H.; Zhang, J. Z. H.; Hou, T. End-point binding free energy calculation with mm/pbsa and mm/gbsa: Strategies and applications in drug design. Chem Rev 2019, 119, 9478-9508, doi: 10.1021/acs.chemrev.9b00055.
27. Pucci, F.; Bernaerts, K.; Teheux, F.; Gilis, D.; Rooman, M. Symmetry principles in optimization problems: An application to protein stability prediction *. IFAC-PapersOnLine 2015, 48, 458-463, doi: https://doi.org/10 16/1 2015.05.068.
28. Guerois, R.; Nielsen, J. E.; Serrano, L. Predicting changes in the stability of proteins and protein complexes: A study of more than 1000 mutations. J Mol Biol 2002, 320, 369-387, doi: 10.1016/s0022-2836 (02) 00442-4.
29. Kellogg, E. H.; Leaver-Fay, A.; Baker, D. Role of conformational sampling in computing mutation-induced changes in protein structure and stability. Proteins 2011, 79, 830-838, doi: 10.1002/prot.22921.
30. Smit, E. Antiviral resistance testing. Current Opinion in Infectious Diseases 2014, 27, 566-572, doi: 10.1097/qco.0000000000000108.
31. Hiv drug resistance database. Available online: https://hivdb.stanford.edu/dr-summary/resistance-notes/NRTI/ (accessed on
32. Amblard, F.; Boucle, S.; Bassit, L.; Chen, Z.; Sari, O.; Cox, B.; Verma, K.; Ozturk, T.; Ollinger-Russell, O.; Schinazi, R. F. Discovery and structure activity relationship of glyoxamide derivatives as anti-hepatitis b virus agents. Bioorg Med Chem 2021, 37, 115952, doi: 10.1016/j.bmc.2020.115952.
33. Jacobson, M. P.; Friesner, R. A.; Xiang, Z.; Honig, B. On the role of the crystal environment in determining protein side-chain conformations. J Mol Biol 2002, 320, 597-608, doi: 10.1016/s0022-2836 (02) 00470-9.
34. Jacobson, M. P.; Pincus, D. L.; Rapp, C. S.; Day, T. J.; Honig, B.; Shaw, D. E.; Friesner, R. A. A hierarchical approach to all-atom protein loop prediction. Proteins: Structure, Function, and Bioinformatics 2004, 55, 351-367.
35. Shelley, J. C.; Cholleti, A.; Frye, L. L.; Greenwood, J. R.; Timlin, M. R.; Uchimaya, M. Epik: A software program for pkaprediction and protonation state generation for drug-like molecules. Journal of Computer-Aided Molecular Design 2007, 21, 681-691, doi: 10.1007/s10822-007-9133-Z.
36. Amblard, F.; Boucle, S.; Bassit, L.; Cox, B.; Sari, O.; Tao, S.; Chen, Z.; Ozturk, T.; Verma, K.; Russell, O.; et al. Novel hepatitis b virus capsid assembly modulator induces potent antiviral responses in vitro and in humanized mice. Antimicrobial Agents and Chemotherapy 2020, 64, doi: 10.1128/AAC.01701-19.
37. Ren, Q.; Liu, X.; Luo, Z.; Li, J.; Wang, C.; Goldmann, S.; Zhang, J.; Zhang, Y. Discovery of hepatitis b virus capsid assembly inhibitors leading to a heteroaryldihydropyrimidine based clinical candidate (gls4). Bioorg Med Chem 2017, 25, 1042-1056, doi: 10.1016/j.bmc.2016.12.017.
38. Günther, S.; Li, B. C.; Miska, S.; Krüger, D. H.; Meisel, H.; Will, H. A novel method for efficient amplification of whole hepatitis b virus genomes permits rapid functional analysis and reveals deletion mutants in immunosuppressed patients. J Virol 1995, 69, 5437-5444, doi: 10.1128/JVI.69.9.5437-5444.1995.
39. In Molecular cloning: A laboratory manual. Cold spring harbor laboratory press. Ny. 16.56-16.67., J., S., F., F. E., T., M., Eds.; Cold Spring Harbor: New York, 1989.
40. Stuyver Lieven, J.; Lostia, S.; Adams, M.; Mathew Judy, S.; Pai Balakrishna, S.; Grier, J.; Tharnish Phillip, M.; Choi, Y.; Chong, Y.; Choo, H.; et al. Antiviral activities and cellular toxicities of modified 2′,3′-dideoxy-2′,3′-didehydrocytidine analogues. Antimicrobial Agents and Chemotherapy 2002, 46, 3854-3860, doi: 10.1128/AAC.46.12.3854-3860.2002.
41. Patel, Dharmeshkumar; Ono, Suzane K.; Bassit, Lcda; Verma, Kiran; Amblard, Frank; Schinazi, Raymond F. Assessment of a Computational Approach to Predict Drug Resistance Mutations for HIV, HBV and SARS-COV-2. Molecules 2022, 27, 5413, 1-14, doi.org/10.3390/molecules27175413.

### Example 5: Mutagenesis of wildtype (NC045512 Wuhan) 3CLpro

Prokaryotic codon optimized SARS-COV-2 3CLpro (GenBank accession NC045512) was generated (Genwiz) and designated wild type. The wild type sequence was used to generate site directed mutations and cloned into the bacterial expression vector pET28A (Millipore). Briefly, PCR using the forward primer ( ) and reverse primer ( ) were used to introduce the mutation. Both mutant PCR fragments were cloned into pET28A.

The tagged protein was purified and validated for enzymatic activity using an assay modeled after the assay disclosed in Loschwitz et al., “Novel inhibitors of the main protease enzyme of SARS-COV-2 identified via molecular dynamics simulation-guided in vitro assay,” Bioorg Chem., 111:104862 (2021), and Owen et al., “An oral SARS-COV-2 Mpro inhibitor clinical candidate for the treatment of COVID-19,” Science, Vol 374, Issue 6575, pp. 1586-1593 (November 2021).

20 μl of 10 μM DABCYL-KTSAVLQSGFRKME-EDANS or the wild type NSP4-5 cleavage sequence fluorogenic substrates (bpsbioscience) were added to 10 ug of Wild type 3CLpro preloaded into a 96 well plate in a 50 ul final volume according to manufacturer's instructions, and fluorescence was measured at the wavelength identified below.

The following protocol was used:
SARS-COV-2 3CLpro Fluorescence Resonance Energy Transfer (FRET) Enzyme Assay
This assay can be used with the SARS-COV-2 3CLpro protease and any inhibitor.
Set-up
Well Recipe (total of 50 μL):
   30 μL diluted enzyme (diluted in buffer)
   10 μL Inhibitor (diluted in DMSO)
   10 μL of substrate (diluted in buffer)
Desired Inhibitor concentration: 0.1-100 μM
Optimized SARS-COV-2 3CLpro concentration: 257 nM
Optimized Substrate concentration: 2 μM

### Experiment

1. Prepare Assay Buffer:
20 mM Tris pH7.3, 100 mM NaCl, 1 mM EDTA, 2 mM DTT, 0.05 mg/mL BSA *Add BSA and DTT day of experiment

2. Prepare Inhibitor Using the Following Equation:

$$( Stock concentration ) ( V 1 ) = ( Desired concentration ) ( 50 / 10 ) ( Desired amount )$$

3. Prepare 257 nM of SARS-COV-2 3CLpro Using the Following Equation:

$$( 183 , 900 nM stock ) ( V 1 ) = 257 nM ) ( 50 / 30 ) ( Desired amount )$$

4. Using a 96-well Corning plate, add 30 μL of 257 nM SARS-COV-2 3CLpro to all experimental wells except the SC control which gets 30 μL of SARS-COV-2 3CLpro buffer instead of protein.

| | | | | | | | | | | 11 | 12 |
|---|---|---|---|---|---|---|---|---|---|---|---|
| 1 | 2 | 3 | 4 | 5 | 6 | 7 | 8 | 9 | 10 | (SC) | (VC) |
| 30 μL | 30 μL | 30 μL | 30 μL | 30 μL | 30 μL | 30 μL | 30 μL | 30 μL | 30 μL | 30 μL | 30 μL |
| Protein | Protein | Protein | Protein | Protein | Protein | Protein | Protein | Protein | Protein | Protein | Buffer |

5. Add 10 μL Inhibitor to wells 1-10. Mix thoroughly using pipette.

6. Add 10 μL DMSO to wells 11-12. Mix thoroughly using pipette.

7. Incubate Protein and Inhibitor for 90 minutes at 25° C.

8. Set up SpectraMax software:
a. Read mode: FL
b. Read type: Kinetic
c. Wavelengths: Excitation 490 nm, Emission Cutoff (select Auto Cutoff) 515 nm, Emission 520 nm.
d. Plate type: 96 Well Corning Half Area opaque.
e. Read area: Select desired read area.
f. Timing: Total run time: 30 minutes, Interval: 5 minutes.
g Shake: 5 seconds before first read, 3 seconds between reads.

9. Prepare 2 μM substrate 10 minutes before 90-minute incubation competition using the following equation;

$$( 447 μM stock ) ( V 1 ) = ( 2 μM ) ( 50 / 10 ) ( Desired amount )$$

10. Add 10 μL of 2 μM substrate to all wells.

11. Mix very well without making bubbles, ideally using a pipette. Insert the plate into the machine. Press Play.

Using this protocol, a protease (PF-07321332 (Pfizer)) was evaluated. The results are discussed below:

IC₅₀: A dose response curve of PF-07321332 (Pfizer) was performed. 5 μM, 2.5 μM, 1.25 μM, 0.625 μM, 0.312 μM, and 0.156 μM PF-07321332 in 10 μL volume was added to 10 μg (20 μL) of Wild type or mutant 3CLpro preloaded into a 96 well plate. 20 μl of 10 μM fluorogenic substrates were added to 50 μl final volume. Fluorescence was read at the wavelengths discussed above.

### Example 6: Assessment of a Computational Approach to Predict Drug Resistance Mutations for HIV, HBV and SARSCOV2

### Abstract:

Viral resistance is a worldwide problem mitigating the effectiveness of antiviral drugs. Mutations in the drug-targeting proteins are the primary mechanism for the emergence of drug resistance. It is essential to identify the drug resistance mutations to elucidate the mechanism of resistance and to suggest promising treatment strategies to counter the drug resistance. However, experimental identification of drug resistance mutations is challenging, laborious and time-consuming. Hence, effective and timesaving computational structure-based approaches for predicting drug resistance mutations are essential and are of high interest in drug discovery research. However, these approaches are dependent on accurate estimation of binding free energies which indirectly correlate to the computational cost. Towards this goal, we developed a computational workflow to predict drug resistance mutations for any viral proteins where the structure is known. This approach can qualitatively predict the change in binding free energies due to mutations through residue scanning and Prime MMGBSA calculations. To test the approach, we predicted resistance mutations in HIVRT selected by ( ) TC and demonstrated accurate identification of the clinical mutations. Furthermore, we predicted resistance mutations in HBV core protein for GLP26 and in SARSCOV2 3CLpro for nirmatrelvir. Mutagenesis experiments were performed on two predicted resistance and three predicted sensitivity mutations in HBV core protein for GLP26, corroborating the accuracy of the predictions.

### 1. Introduction

The discovery of effective antiviral drugs revolutionized world health by delaying virus-associated disease progression and thus saving millions of lives. Despite these medical advances, the selection of drug-resistant strains is a persistent problem that leads to viral breakthrough and reduced antiviral efficacy [1-4]. There are several mechanisms reported for the development of drug resistance [5,6]. Random mutations in viral genes which alter the binding of the drug to its protein target are the primary mechanism of acquired drug resistance in viruses [6]. The mutation rate in RNA viruses is generally very high-estimated at 10-4 per nucleotide per replication. In contrast, DNA viruses have an estimated mutation rate of 10-8 per nucleotide per replication [7,8]. The primary cause of failure of anti-HIV therapy is the selection of drug-resistant mutants. With the advent of genetic sequencing and a deeper understanding of drug resistance mechanisms, combination drug therapy has become the standard of care [9,10]. Similarly, the use of multiclass combination therapy in HCV effectively prevented the selection of resistant mutants, leading to curative rates in the range of 98% [11]. Thus, the emergence of drug-resistant viruses is one of the greatest risks to public health and is a priority across the globe. Foreknowledge of potential mutations that could be selected in vitro and in vivo, coupled with a comprehensive understanding of viral resistance mechanisms, is of immense importance in developing more effective and durable drug treatments.

Experimentally, drug-resistant viruses are typically selected by maintaining an infected culture under drug pressure for months (sometimes years) without a guarantee that resistance will emerge under those specific cellular conditions [12]. In certain situations, resistance appears exclusively in clinical settings, requiring hasty characterization of the mutation and viral species [13]. The ability to predict drug resistance mutations expedites understanding of antiviral efficacy, anticipates activity against existing mutant strains, delivers mechanistic insight into how specific mutants confer resistance, allows for the design of drug combinations that are not cross-resistant, forecasts mutant species that may develop in clinical settings, provides guidance on the development of diagnostic assays that detect mutations and generally provides broad utility and benefit to infectious disease drug discovery [14].

Numerous efforts have been made to develop tools to predict drug resistance mutations. One group of prediction models includes sequence-based approaches, which use various machine learning methods. These prediction models rely primarily on primary sequences of the protein or genotypic sequence data, and their prediction accuracies are dependent on the availability of large and diverse training sets [15-18]. The main advantage of these methods is that they are computationally efficient. A weakness of these methods is that they are reliant on the availability of training set data. Further, without 3D structural information and knowledge of the enzymatic function of the mutated residues, this group of models fails to link viral genetic mutations and structural changes due to corresponding phenotypic mutations [14,19,20]. A second type of prediction models is based on the 3D structure of the target proteins. In the last few decades, the availability of a large number of 3D structures of protein targets has enabled the implementation of various structure-based molecular modeling approaches to study binding interactions and binding free energies of drug molecules with their corresponding protein targets. The binding free energies are crucial for facilitating the prediction of drug resistance mutations [21-24]. Although these methods are advantageous over sequence-based methods, they can be time-consuming or show low predictive accuracy. Hence, there is a need for novel structure-based methods with an optimum balance between computational efficiency and accuracy [25].

Molecular mechanics-generalized Born surface area (MMGBSA) is one of the structure-based approaches widely used to estimate binding affinities in protein-ligand or protein-protein complexes [26-29]. Recently, Schrödinger utilized a physics-based scoring function together with the MMGBSA model (Prime MMGBSA) to calculate changes in the binding free energy of protein-protein complexes due to single point mutations, which was called residue scanning [26,27]. Moreover, it was also shown that Prime MMGBSA has slightly better accuracy compared to other prediction methods such as PoPMuSiCsyn [30], FoldX and Rossetta in predicting binding affinities due to single point mutations in protein-protein complexes [26,27]. Although promising, the ability of Prime MMGBSA to calculate binding affinity due to single amino acid mutations and to predict resistance mutations based on interactions between small molecules and their protein targets has not been explored thoroughly.

Typically, drugs-elected resistance mutations in viruses meet three requirements: (1) a decrease in the inhibitor binding affinity, (2) retention of the native substrate binding affinity to maintain essential viral function and (3) accessibility via a single nucleotide substitution (SNS) in the wild-type codon [19,33,34]. Very few active-site mutations meet all three criteria, providing the potential to efficiently predict resistance mutations. Moreover, these criteria can be analyzed computationally using structure-based methods. Herein, we describe the prediction of the binding affinities with the help of two Schrödinger suite modules, Residue Scanning and Prime MMGBSA, in the context of predicting mutations conferring resistance to small molecule drugs following single amino acid mutations. We first implemented and validated this approach by predicting known resistance mutations for the approved HIV reverse transcriptase (RT) inhibitor ( ) TC. Second, we used the approach to predict resistance mutations associated with GLP26, a known HBV capsid assembly modulator (CAM) 26, and with nirmatrelvir, an emergency FDA-approved SARSCOV2 3CL protease inhibitor. Five GLP26 resistance and sensitivity mutations were validated in mutagenesis experiments.

### 2. Results and Discussion

The purpose of this study was to assess the prediction accuracy of drug resistance mutation identification using the computational workflow shown in FIG. 1. This workflow utilizes both residue scanning and Prime MMGBSA calculations with the goal of optimizing the balance between predictive accuracy and computational efficiency. This approach was used on three protein-drug complexes: (1) HIV RT complexed with ( )FTCTP, (2) HBV core protein complexed with GLP26 and (3) SARSCOV2 3CLpro complexed with the protease inhibitor nirmatrelvir.

The approach began with residue scanning followed by Prime MMGBSA calculations (FIG. 14). Residue scanning generated mutations for specified residues using the Prime rotamer search algorithm. We then performed Prime MMGBSA refinement of the bound and unbound state for each system for both wild-type and mutant protein structures. We kept the protein backbone and the neighboring side chains fixed, allowing for rapid screening of the mutations and predicted binding affinities in a computationally efficient way. The main goal of residue scanning was to filter out mutations with increased drug/substrate binding affinities (ΔΔG<0 kcal/mol) early on and to keep only mutations with a decrease in binding affinities (ΔΔG>0 kcal/mol), allowing binding affinities with sidechain flexibility in the binding sites to be explored using Prime MMGBSA at a later time point. Mutations with increasing binding affinities (ΔΔG<0 kcal/mol) are in energy minimum conformations [26], and we hypothesized that incorporating sidechain flexibility would be less likely to decrease binding affinities, and to therefore have less probability of changing binding free energies from a negative value (ΔΔG<0 kcal/mol) to a positive value (ΔΔG>0 kcal/mol). Moreover, mutations with ΔΔG>0 kcal/mol for the drug complexes and ΔΔG≤0 kcal/mol for the native substrate complexes (i.e., decreasing binding affinities for the drug molecules while maintaining or increasing binding affinities for substrates) are targeted as they could be potential resistance mutations.

In the next step, Prime MM-GBSA calculations were implemented with side-chain flexibility to calculate drug/substrate binding affinities with their point-mutated protein targets. Point-mutated structures, in which the mutations have ΔΔG>0 kcal/mol for drugs/substrates, were identified from the residue scanning step. Side-chain flexibility was provided for the residues located within 8 Å of the drugs/substrates to explore the conformational space of the side chains within the binding sites due to point mutations. Moreover, binding affinities for mutations with ΔΔG>0 kcal/mol could be improved from ΔΔG>0 kcal/mol to ΔΔG≤0 kcal/mol by incorporating side-chain flexibility in the binding sites. With Prime MM-GBSA, the binding affinity (ΔG) of a drug/substrate with wild-type and mutant protein targets is calculated separately to determine a free energy change (ΔΔG). Mutations that maintain/increase binding affinities of the substrates (ΔΔG≤0 kcal/mol) and decrease binding affinities of the drug molecules (ΔΔG>0 kcal/mol) are potential drug resistance mutations. Most antiviral drugs are known to have low genetic barriers, which means that viruses can become resistant through non-synonymous single-nucleotide polymorphism (SNP). Therefore, amino acid mutations associated with single-nucleotide polymorphisms were prioritized as possible drug resistance mutations.

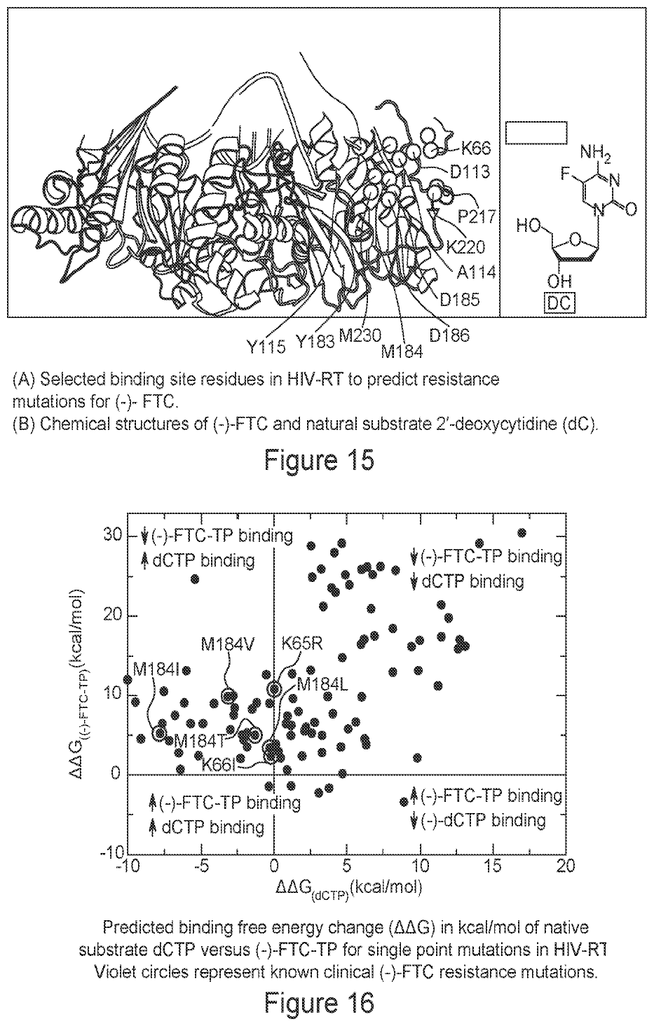

To evaluate our approach, we used (-)-FTC (emtricitabine), which in its 5′-triphophate form ((-)-FTC-TP) is a well-characterized HIV reverse transcriptase (HIV-RT) inhibitor, to determine if we were able to recapitulate its mutation profile. HIV-RT polymerizes the viral DNA primer from an RNA template. To do so, the active site binds 2′-deoxynucleotide triphosphates, such as 2′-deoxcytidine triphosphate dCTP (FIG. 15), for chemical incorporation into the growing DNA strand [35]. Nucleoside analogs have been developed that bind to HIV-RT and terminate DNA chain elongation after incorporation. (-)-FTC is a frontline nucleoside analog in antiretroviral therapy [36-38]. The drug is converted to the active nucleoside triphosphate form by host kinases, and the active nucleoside triphosphate form then outcompetes dCTP for binding to HIV-RT and terminates genome chain polymerization. The pharmacological activities and resistance mutations of (-)-FTC were first described and studied rigorously by Schinazi et al. [39,40]. Moreover, the clinically significant resistance mutations are reported and well-studied [41]. It is well established that (-)-FTC selects the M184V resistance mutation in the HIV-RT active site leading to virologic breakthrough [39,40]. Thus, HIV RT with (-)-FTC was the ideal system for testing the ability of our computational protocol to predict the resistance mutations.

The approach predicted 157 resistance mutations through the first step of residue scanning and 48 resistance mutations through the second step of Prime MM-GBSA calculations. This demonstrates that incorporation of side-chain flexibility in Prime MM-GBSA filtered out mutations that do not reduce drug/substrate binding affinities and that resistance mutations were selected. Finally, SNP mutations were selected as probable resistance mutations, as shown in FIG. 16 and the following table:

Predicted resistance mutations in HIV-RT for (−)-FTC.

| WT | Predicted Resistance Mutations |
|---|---|
| M184 | V, I, A, R, N, D, C, Q, G, L, K, P, S, T, W, Y |
| I63 | D, E |
| K65 | R |
| K66 | I, P, T, G |
| D113 | E |
| Y115 | A, R, N, D, C, Q, E, G, H, I, L, K, P, S, T, V |
| Q151 | E, I, C, D, P |
| G152 | D, E |
| Y183 | D, E |

Bold—clinically reported (−)-FTC resistance mutations in HIV-RT.

FIG. 16 shows the predicted binding free energy changes of natural substrate dCTP (ΔΔG(dCTP)) versus drug (-)-FTC-TP (ΔΔG(FTC-TP)) obtained from Step 2. Mutations leading to a decrease in binding affinity of (-)-FTC-TP while maintaining or increasing the binding affinity of substrate dCTP are shown on the top left side of the graph. Thus, mutations in this region could be potential drug resistance mutations. This approach allowed us to recapture several clinically relevant resistance mutations, including M184V and M184I, as shown in FIG. 16 and the previous table, two mutations known to show 500-1000-fold resistance to (-)-FTC. Although the predicted AAG values do not correlate with experimental values, the predicted AAG values for M184V and M184I are higher relative to other mutations. Between M184V and M184I, the AAG value of M184V is higher than that of M184I, which corroborates the experimental data.

Moreover, four other clinically reported mutations, M184L, M184T, K65R and K66I, were also predicted using our approach, validating the accuracy of our method. However, it is worth noting that our model did not predict the Q151M mutation, a clinically known mutation to reduce 2-fold EC50 of (-)-FTC. Enzymatically, the Q151M mutation remains sensitive to (-)-FTC with the same activity [42], and our approach is based on target protein structure, providing a possible explanation for why this mutation was not identified through our approach. Although Q151M was not predicted, other Q151 mutations Q151E/I/C/D/P were predicted as resistance mutations for (-) -FTC. Other predicted resistance mutations from this table have not yet been reported, but they could be put on a potential watchlist for resistance to (-)-FTC. The prioritized list of resistance mutations for (-)-FTC based on AAG values is summarized in supporting information.

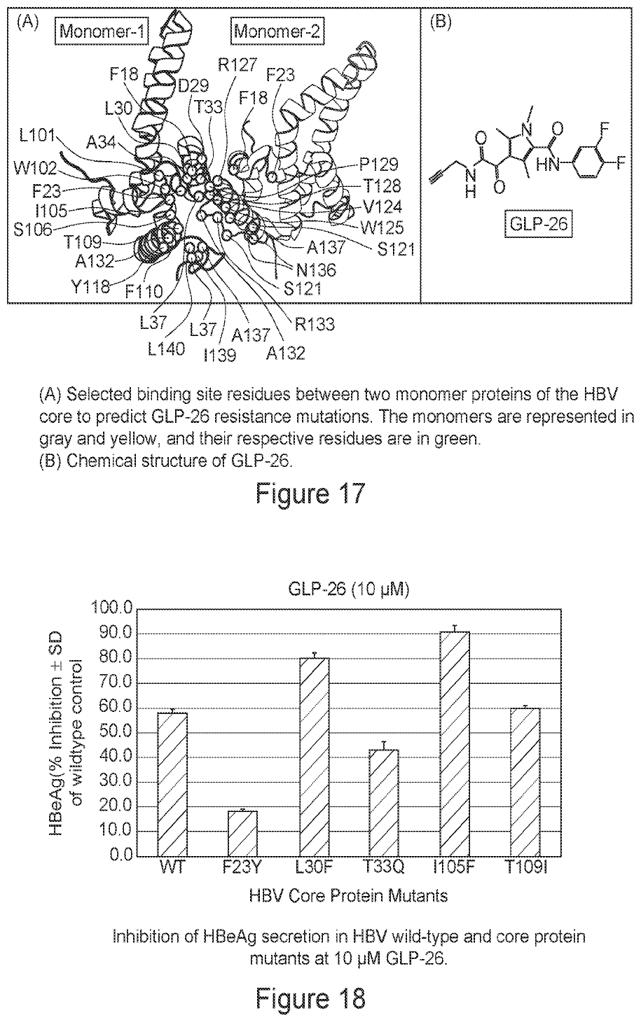

To explore the predictive approach further, attention was turned to the HBV capsid, which plays a pivotal role in the replication cycle of the virus. Capsid assembly modulators (CAMs) have been shown to bind between two monomeric core proteins and impair capsid assembly by affecting the kinetics and/or the binding strength be-tween core protein dimers [43]. GLP-26 is a non-toxic, highly potent HBV CAM which displays promising effects in vitro and in various animal models [44-46]. Based on the unique profile of this compound, we decided to use our approach to predict its resistance mutation profile. As there is no substrate involved in the HBV capsid assembly, AAG values of two monomeric core proteins due to mutations were compared with and without GLP-26 complexes. The selected binding site residues from both monomers are shown in FIG. 17A.

Using the three-step workflow described herein, a series of resistance mutations in HBV core protein was predicted for GLP-26, and these mutations are shown in the following table:

Predicted resistance mutations in HBV core protein for GLP-26.

| WT | Predicted Resistance Mutations |
|---|---|
| A132 | Y, W, V, S |
| S106 | W, Y, F, L, R, C |
| L140 | P, C, I, V, H, M, Y |
| T128 | K, R, P, I, M |
| L30 | W, H, R |
| W102 | G, C, L, Y, F, S, R |
| Y118 | Q, D, C, W, E, H, L, K, R, F |
| P134 | L, H, S |
| R133 | I, C, L, T, H, M, G |
| W125 | G |
| T33 | R, M, Q, I |
| F110 | I, L, M |
| P25 | L, Q, R |
| F23 | M, L, Y |
| I105 | M, F, S, V, T |
| P138 | H, A |
| P129 | H, T, S, Q |
| F122 | V, L, C |
| N136 | R |
| V124 | G, A |
| L37 | P, Y, Q |
| R127 | Q |
| A34 | E |

Bold—predicted resistance mutations in HBV core protein for GLP-26 with ΔΔG > 3 kcal/mol.

Interestingly, the F110I, T128I and L140I mutations have been reported for other CAMs [47], F101I (JNJ-6379 and Bay41-4109), T128I and L140I (JNJ-6379). These mutations were predicted to reduce GLP-26 binding affinity to a higher degree (ΔΔG>3 kcal/mol). Other known CAM-associated mutations including F23Y, T33Q, L37Q, I105T, I105V, Y118F, V124A and V124G have also been reported [47], but are predicted to show only mild to moderate effect on the binding of GLP-26. Finally, T109 mutations, known to be resistant to most HBV CAMs [47,48], are not predicted to be an issue with GLP-26 (FIG. 4 and the previous table) and could, therefore, provide options for combination therapies with other CAMs. To validate our prediction model and its accuracy, we performed site-directed mutagenesis experiments on HBV core for mutations F23Y, L30F, T33Q, I105F and T109I and evaluated GLP-26 against these HBV core protein mutants. F23Y and L30F are resistance mutations for CAMs JNJ-6379 and BAY41-4109, while T33Q is a resistance mutation for SBA_R01, BAY41-4109 [47]. Thus, the selection of these five mutations covered the sensitivity and resistance for GLP-26 and novelty with respect to the resistance profile for other CAMs. GLS4, a well-known CAM, is resistant to T109I [47], so we tested GLS4 against T109I to validate our experiment.

The effect of GLP-26 on HBeAg production (EC₅₀) was measured (FIG. 5) at 10 μM concentration. HBeAg production is a rapid and direct marker that is affected by capsid effector modulators in this transfection assay and is also largely cccDNA-dependent, and therefore can serve as a surrogate marker for cccDNA [49,50]. Interestingly, we were able to recapture the mutation profile predicted by our modeling approach, as shown in the following table. GLP-26 was shown to be active against L30F, I105F and T109I mutants while T33Q and F23Y significantly decreased the GLP-26 effect on HBeAg production.

### Experimental Results for the Selected Mutations in HBV Core Protein for GLP-26.

| HBV Core Protein Mutants | GLP26 | Predictions for GLP26 |
|---|---|---|
| WT | Sensitive | — |
| F23Y | Resistant | Resistant |
| L30F | Sensitive | Sensitive |
| T33Q | Resistant | Resistant |
| I105F | Sensitive | Sensitive |
| T109I | Sensitive | Sensitive |

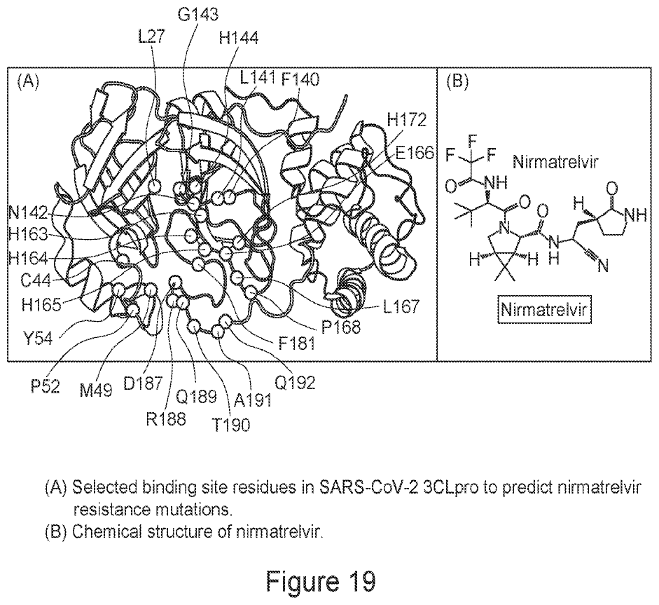

Finally, we applied our prediction approach to SARS-COV-2 3CLpro and the recently approved nirmatrelvir, which should be helpful in rapid assays to diagnose resistance and to select additional non-cross-resistant protease inhibitors. 3CLpro, one of the major therapeutic targets for anti-SARS-COV-2 drugs, plays an important role in viral replication and cleaves polyprotein chains into non-structural proteins (NSPs). NSP peptide chains are the native substrates for 3CLpro. Thus, 3CLpro has 11 substrate peptides, and 3-D structures of six of them had been reported in Protein Data Bank (https://www.rcsb.org) complexed with 3CLpro when we started the work. The binding site residues of 3CLpro involved in this study are shown in FIG. 19A.

Mutations were considered resistance mutations if they decreased nirmatrelvir binding affinity (ΔΔG>0 kcal/mol) but maintained or increased the binding affinity (ΔΔG≤0 kcal/mol) for at least three out of the six NSP substrates. The mutations identified using our approach are summarized in the following table, and the list of prioritized resistance mutations is provided in supporting information.

### Predicted Resistance Mutations in SARS-COV-2 3CLpro for Nirmatrelvir.

| WT | Predicted Resistance Mutations |
|---|---|
| S144 | Y, W |
| H163 | Y, Q |
| L167 | Q, P, F, R, I, M, H |
| T190 | N, I, R, K, A |
| R188 | H, K, N, L |
| M49 | N, R, K, F |
| P168 | S, Q, R, A, H |
| Q192 | H, K, Y, L, P |
| E166 | G, A, K, V, N |
| F140 | S, I, V, L |
| G143 | A, C |
| Y54 | D, C, E, Q, N, F, G, H, M, S, W, K |
| P52 | R, S |
| H172 | Q, N |
| H164 | N, L, D, Q, K, T, E |
| Q189 | H |
| A191 | G, D |
| D187 | G, Q, A |
| L141 | Q, H |
| M165 | L |
| F181 | L, I, V, S, C |
| N142 | I |

Bold—predicted resistance mutations in SARS-CoV-2 3CLpro for nirmatrelvir with ΔΔG > 3 kcal/mol.

It is worth noting that these mutations were analyzed with a genomic database (https://www.gisaid.org) to determine if any of these are known without drug treatment. Interestingly, Y54C was a mutant reported in March 2020 in Malaysia. Further, T190I has been reported 110 times from 15 different countries and represents 0.03% of the sequenced NSP5. As of now, there are no experimental or clinical resistance mutations reported in peer-reviewed articles on nirmatrelvir, but if our predictions are correct, these two naturally occurring variants could emerge with the use of nirmatrelvir and pose a severe threat to the population. We are currently evaluating the effect of the mutations on the binding of nirmatrelvir biochemically since our laboratory cannot perform gain-of-function studies with live coronaviruses.

### 3. Materials and Methods

### 3.1. Test System Selection and Preparation

HIV RT complexed with (-)-FTC-TP (PDB ID-6UJX), natural substrate and dCTP (PDB ID-6UIT) were selected to assess the approach to predict the resistance mutations. The crystal structure of GLP-26 with HBV core protein has not been resolved; therefore, our previously published modeled complex of GLP-26 with HBV core protein was selected for this study [44]. GLP-26 binds between two dimeric subunits and so tetramer HBV core protein (PDB ID-1QGT) was used. One additional system, SARS-COV-2 3CLpro for nirmatrelvir (PDB ID-7RFS) was used to predict the resistance mutations. For the substrates, 3-D structures of SARS-COV-2 3CLpro complexed with nsp4-nsp5 (PDB ID-7N89), nsp6-nsp7 (PDB ID-7DVX), nsp8-nsp9 (PDB ID-7MGR), nsp9-nsp10 (PDB ID-7DVY, nsp14-nsp15 (PDB ID-7DW6) and nsp15-nsp16 (PDB ID-7DW0) were used.

The PDB structures were prepared using Protein Preparation Wizard in Maestro (Schrödinger Release 2020-4; Schrödinger). Missing residues and loops were added and minimized using Prime [53,54]. Crystallographic waters were deleted, and the hydrogen bonding network was optimized using Epik at neutral pH [55]. The final structures were minimized with heavy atom restraints using the OPLS3e force field. The minimization was terminated when the heavy-atom root mean square deviation reached 0.3 Å.

### 3.2. Residue Scanning

The binding site residues of drug/substrate were defined by Binding Site object in Maestro (Schrödinger Release 2020-4; Schrödinger). The change in binding affinities of the residues due to mutations was calculated using Residue Scanning module in BioLuminate (Schrödinger Release 2020-4; Schrödinger). Before residue scanning, a new chain ID was generated for drug/substrate/ligand molecule. In the residue scanning panel, all (allowed) mutations of interest residue were generated, and “Stability and Affinity” calculation was performed between drug/substrate/ligand chain and other binding partnered protein chains. During the calculations, for the refinement of the mutated residue, “Side-chain prediction with backbone sampling” option was selected with a cutoff of 0.0 Å. The residue scanning uses MM-GBSA refinement without any side-chain and backbone flexibility.

### 3.3. Prime MM-GBSA Calculations

The WT-drug/substrate and MUT-drug/substrate complexes which showed greater than 0 kcal/mol binding affinity change in residue scanning calculations were selected for Prime MM-GBSA estimation with side-chain flexibility. The protein complexes generated from residue scanning were split into ligand and protein structures which were selected for Prime MM-GBSA calculations. For the covalent systems, the covalent bond was removed for Prime MM-GBSA calculations. VSGB (variable-dielectric generalized Born) solvation model and OPLS3e force field were utilized during Prime MM-GBSA calculations. Side-chain flexibility was incorporated for the residues within 8 Å of the drug/substrate/ligand molecule by selecting a distance from ligand of 8 Å. For the sampling, the “minimize” option was selected. The changes in binding affinities, ΔG^{MUT} and ΔG^{WT}, were calculated separately by Prime MM-GBSA, and the difference between them was used to calculate the change in the binding affinity due to mutations:

$$Δ Δ {G}_{bind} = Δ {G}^{MUT} - Δ {G}^{WT} \qquad ( 1 )$$

### 3.4. Cell Lines

Wild-type HBV DNA was amplified and cloned as previously described (38, 39). Five HBV core mutants (F23Y, L30F, T33Q, I105F and T109I) were created by substituting nucleotides to change the codon as indicated below using the QuikChange II Site-Directed Mutagenesis Kit (Agilent, Santa Clara, CA, USA). Primers used for site-directed PCR mutagenesis are described in Table 5. The core genes of the mutants were sequenced bidirectionally by GENEWIX (New Jersey, USA) to confirm the introduction of mutations.

HepNTCP-DL cells were maintained in Dulbecco's modified minimal essential medium (DMEM) supplemented with 10% FBS and 0.1 mM non-essential amino acids (NEAA).

### 3.5. Compound Synthesis

GLP-26 and GLS4 were prepared in-house according to published procedures [45,56]. Both compounds had a purity of >95% as determined by 1H, 13C, 19F nuclear magnetic resonance (NMR) and high-pressure liquid chromatography (HPLC) analysis. Entecavir (ETV) was purchased from commercial vendors and confirmed at >95% purity using standard analytical methods such as mass spectrometry and NMR.

3.6. Transfection of Full-Length HBV DNA into HepNTCP-DL Cells.

Full-length HBV DNA wild-type and core mutants were prepared for transfection as previously described [57]. HepNTCP-DL cells were seeded in either 96- or 24-well collagen-coated plates in DMEM supplemented with 10% FBS and 0.1 mM NEAA and maintained in a tissue culture incubator at 37° C. with 5% CO₂. The cells were 90% confluent the next day, and the medium was changed to DMEM supplemented with 3% FBS and 0.1 mM NEAA. Transfection of HBV DNA was performed with Lipofectamine 3000 reagent (Invitrogen, Carlsbad, CA, United States) according to the manufacturer's instructions. Twenty-four hours after transfection, the medium was replenished with drug-free medium or medium containing different concentrations of either GLP-26 or GSL4. Medium and cells (rinsed 3 times with ice-cold PBS) were harvested 3 days later. The efficiency of transfection was monitored by co-transfecting a β-galactosidase expression plasmid, pCMVB (CLONTECH Laboratories Inc., Palo Alto, California, USA). Assays for β-galactosidase in extracts of HuH-7 cells were performed as described [58]. Experiments were performed in triplicate.

### 3.7. Analysis of HBV HBeAg Production

Levels of HBeAg secreted in the culture medium were measured by using an HBsAg or HBeAg enzyme-linked immunosorbent assay (ELISA) kit (BioChain Institute Inc. Hayward, CA), according to the manufacturer's protocol. The concentration of compound that reduced levels of secreted HBeAg by 50% (EC₅₀) was determined by linear regression.

TABLE X4
Primers used for site-directed PCR
mutagenesis of HBV core.

| Pair Name | Sequence Information |
|---|---|
| core T109I | 5′-ctataactgtttctcttccaaaaatgagacaaga |
| mutant c326t | aatgtgaaaccac-3′ |
| | 5′-tgtgtttcacatttcttgtctatttttggaagag |
| | aaacagttatag-3′ |
| core I105F | 5′-ccaaaagtgagacaagaaaagtgaaaccacaaga |
| mutant a313t | gttgc-3′ |
| | 5′-gcaactcttgtggtttcacttttcttgtctcact |
| | tttgg-3′ |
| core T33Q | 5′-atacagagctgaggcctgatctagaagatctcgt |
| mutant | actgaaggaaaga-3′ |
| a97c_c98a_ | 5′-tctttccttcagtacgagatcttctagatcaggc |
| c99g | ctcagctctgtat-3′ |
| core L30F | 5′-gcggtatctagaaaatctcgtactgaaggaaaga |
| mutant c88t | agtc-3′ |
| | 5′-gacttctttccttcagtacgagattttctagata |
| | ccgc-3′ |
| core F23Y | 5′-tctcgtactgaaggaaagtagtcagaaggcaaaa |
| mutant t68a | acg-3′ |
| | 5′-cgtttttgccttctgactactttccttcagtacg |
| | aga-3′ |
| core T109I | 5′-ctataactgtttctcttccaaaaatgagacaaga |
| mutant c326t | aatgtgaaaccac-3′ |

### 4. Conclusions

In this study, we assessed the ability of a computational approach containing residue scanning and prime MM-GBSA calculations to predict resistance mutations with an optimum balance between computational efficiency and accuracy. The approach successfully validated the prediction of the resistance mutations in HIV-RT for (-)-FTC-TP and can be used to predict the resistance mutations in HBV core protein for GLP26 and in SARS-COV-2 3CLpro for nirmatrelvir. Three sensitivity mutations, L30F, I105F and T109I, and two resistance mutations, F23Y and T33Q, in HBV core protein for GLP26 were studied experimentally and validated our predictions. Hence, the approach demonstrated a strong correlation between prediction and experimental findings. Even though there are still areas for improvement such as accurately estimating binding affinity for the prioritization of resistance mutations, and predicting multiple drug resistance mutations, our approach could be used to develop drug treatment strategies for different antiviral agents, taking into account potential mutations that could arise and determining ways to minimize their selection in culture and in humans.

### REFERENCES

1. Perrin, L.; Telenti, A. HIV treatment failure: Testing for HIV resistance in clinical practice. Science 1998, 280, 1871-1873. https://doi.org/10.1126/science.280.5371.1871.
2. Elhence, A.; Singh, A.; Anand, A.; Kumar, R.; Ashraf, A.; Kumar, S.; Pradhan, D.; Pathak, P.; Vaishnav, M.; Rajput, M. S.; et al. Real-world re-treatment outcomes of direct-acting antiviral therapy failure in patients with chronic hepatitis C. J. Med. Virol. 2021, 93, 4982-4991. https://doi.org/10.1002/jmv.26971.
3. Stuyver, L. J.; Locarnini, S. A.; Lok, A.; Richman, D. D.; Carman, W. F.; Dienstag, J. L.; Schinazi, R. F. Nomenclature for antiviral-resistant human hepatitis B virus mutations in the polymerase region. Hepatology 2001, 33, 751-757. https://doi.org/10.1053/jhep.2001.22166.
4. Burrel, S.; Topalis, D.; Boutolleau, D. Herpes simplex virus resistance to antivirals. Virologie 2020, 24, 325-342. https://doi.org/10.1684/vir.2020.0864.
5. Borst, P. Genetic mechanisms of drug resistance. A review. Acta Oncol. 1991, 30, 87-105. https://doi.org/10.3109/02841869109091819.
6. Erickson, J. W.; Burt, S. K. Structural mechanisms of HIV drug resistance. Annu. Rev. Pharmacol. Toxicol. 1996, 36, 545-571. https://doi.org/10.1146/annurev.pa.36.040196.002553.
7. Mason, S.; Devincenzo, J. P.; Toovey, S.; Wu, J. Z.; Whitley, R. J. Comparison of antiviral resistance across acute and chronic viral infections. Antivir. Res. 2018, 158, 103-112. https://doi.org/10.1016/j.antiviral.2018.07.020.
8. Vere Hodge, A.; Field, H.J. General mechanisms of antiviral resistance. In Genetics and Evolution of Infectious Disease; Elsevir: Amsterdam, Netherlands, 2011; pp. 339-362. https://doi.org/10.1016/B978-0-12-384890-1.00013-3.
9. Amblard, F.; Patel, D.; Michailidis, E.; Coats, S.; Kasthuri, M.; Biteau, N.; Tber, Z.; Ehteshami, M.; Schinazi, R. F. HIV nucleoside reverse transcriptase inhibitors. Eur. J. Med. Chem. 2022, 240, 114554. https://doi.org/10.1016/j.ejmech.2022.114554.
10. Schinazi, R. Combined chemotherapeutic modalities for viral infections: Rationale and clinical potential. In Synergism and Antagonism in Chemotherapy; Chou, T. C., Rideout, D. C., Eds.; Academic Press, Orlando, FL, 1991; Chapter 4, pp. 109-181.
11. Schinazi, R.; Halfon, P.; Marcellin, P.; Asselah, T. HCV direct-acting antiviral agents: The best interferon-free combinations. Liver Int. 2014, 34, 69-78.
12. Vacca, J. P.; Dorsey, B. D.; Schleif, W. A.; Levin, R. B.; McDaniel, S. L.; Darke, P. L.; Zugay, J.; Quintero, J. C.; Blahy, O. M.; Roth, E.; et al. L-735,524: An orally bioavailable human immunodeficiency virus type 1 protease inhibitor. Proc. Natl. Acad. Sci. USA 1994, 91, 4096-4100. https://doi.org/10.1073/pnas.91.9.4096.
13. Larder, B. A.; Kemp, S. D. Multiple mutations in HIV-1 reverse transcriptase confer high-level resistance to zidovudine (azt). Science 1989, 246, 1155-1158. https://doi.org/10.1126/science.2479983.
14. Cao, Z. W.; Han, L. Y.; Zheng, C. J.; Ji, Z. L.; Chen, X.; Lin, H. H.; Chen, Y. Z. Computer prediction of drug resistance mutations in proteins. Drug Discov. Today 2005, 10, 521-529. https://doi.org/10.1016/S1359-6446 (05) 03377-5.
15. Steiner, M. C.; Gibson, K. M.; Crandall, K. A. Drug resistance prediction using deep learning techniques on HIV-1 sequence data. Viruses 2020, 12, 560. https://doi.org/10.3390/v12050560.
16. Beerenwinkel, N.; Däumer, M.; Oette, M.; Korn, K.; Hoffmann, D.; Kaiser, R.; Lengauer, T.; Selbig, J.; Walter, H. Geno2pheno: Estimating phenotypic drug resistance from HIV-1 genotypes. Nucleic Acids Res. 2003, 31, 3850-3855. https://doi.org/10.1093/nar/gkg575.
17. Riemenschneider, M.; Hummel, T.; Heider, D. Shiva-A web application for drug resistance and tropism testing in HIV. BMC Bioinform. 2016, 17, 314. https://doi.org/10.1186/s12859-016-1179-2.
18. Bonet, I. Machine learning for prediction of HIV drug resistance: A review. Curr. Bioinform. 2015, 10, 579-585.
19. Khalid, Z.; Sezerman, O.U. Prediction of HIV drug resistance by combining sequence and structural properties. IEEE/ACM Trans. Comput. Biol. Bioinform. 2018, 15, 966-973. https://doi.org/10.1109/TCBB.2016.2638821.
20. Weber, I. T.; Harrison, R. W. Tackling the problem of HIV drug resistance. Postepy Biochem. 2016, 62, 273-279.
21. Jenwitheesuk, E.; Samudrala, R. Prediction of HIV-1 protease inhibitor resistance using a protein-inhibitor flexible docking approach. Antivir. Ther. 2005, 10, 157-166.
22. Toor, J. S.; Sharma, A.; Kumar, R.; Gupta, P.; Garg, P.; Arora, S. K. Prediction of drug-resistance in HIV-1 subtype c based on protease sequences from art naive and first-line treatment failures in north india using genotypic and docking analysis. Antivir. Res. 2011, 92, 213-218. https://doi.org/10.1016/j.antiviral.2011.08.005.
23. Hou, T.; Yu, R. Molecular dynamics and free energy studies on the wild-type and double mutant HIV-1 protease complexed with amprenavir and two amprenavir-related inhibitors: Mechanism for binding and drug resistance. J. Med. Chem. 2007, 50, 1177-1188. https://doi.org/10.1021/jm0609162.
24. Agniswamy, J.; Louis, J. M.; Roche, J.; Harrison, R. W.; Weber, I. T. Structural studies of a rationally selected multi-drug resistant HIV-1 protease reveal synergistic effect of distal mutations on flap dynamics. PLOS ONE 2016, 11, e0168616. https://doi.org/10.1371/journal.pone.0168616.
25. Hao, G. F.; Yang, G. F.; Zhan, C.G. Structure-based methods for predicting target mutation-induced drug resistance and rational drug design to overcome the problem. Drug Discov. Today 2012, 17, 1121-1126. https://doi.org/10.1016/j.drudis.2012.06.018.
26. Beard, H.; Cholleti, A.; Pearlman, D.; Sherman, W.; Loving, K. A. Applying physics-based scoring to calculate free energies of binding for single amino acid mutations in protein-protein complexes. PLOS ONE 2013, 8, e82849. https://doi.org/10.1371/journal.pone.0082849.
27. Duan, J.; Lupyan, D.; Wang, L. Improving the accuracy of protein thermostability predictions for single point mutations. Biophys. J. 2020, 119, 115-127. https://doi.org/10.1016/j.bpj.2020.05.020.
28. Genheden, S.; Ryde, U. The MM/PBSA and MM/GBSA methods to estimate ligand-binding affinities. Expert Opin. Drug Discov. 2015, 10, 449-461. https://doi.org/10.1517/17460441.2015.1032936.
29. Wang, E.; Sun, H.; Wang, J.; Wang, Z.; Liu, H.; Zhang, J. Z. H.; Hou, T. End-point binding free energy calculation with MM/PBSA and MM/GBSA: Strategies and applications in drug design. Chem. Rev. 2019, 119, 9478-9508. https://doi.org/10.1021/acs.chemrev.9b00055.
30. Pucci, F.; Bernaerts, K.; Teheux, F.; Gilis, D.; Rooman, M. Symmetry principles in optimization problems: An application to protein stability prediction. IFAC-Pap. 2015, 48, 458-463. https://doi.org/10.1016/j.ifacol.2015.05.068.
31. Guerois, R.; Nielsen, J. E.; Serrano, L. Predicting changes in the stability of proteins and protein complexes: A study of more than 1000 mutations. J. Mol. Biol. 2002, 320, 369-387. https://doi.org/10.1016/s0022-2836 (02) 00442-4.
32. Kellogg, E. H.; Leaver-Fay, A.; Baker, D. Role of conformational sampling in computing mutation-induced changes in protein structure and stability. Proteins 2011, 79, 830-838. https://doi.org/10.1002/prot.22921.
33. Smit, E. Antiviral resistance testing. Curr. Opin. Infect. Dis. 2014, 27, 566-572. https://doi.org/10.1097/qco.0000000000000108.
34. Irwin, K. K.; Renzette, N.; Kowalik, T. F.; Jensen, J.D. Antiviral drug resistance as an adaptive process. Virus Evol. 2016, 2, vew014. https://doi.org/10.1093/ve/vew014.
35. Arnold, E.; Das, K.; Ding, J.; Yadav, P. N.; Hsiou, Y.; Boyer, P. L.; Hughes, S.H. Targeting HIV reverse transcriptase for anti-aids drug design: Structural and biological considerations for chemotherapeutic strategies. Drug Des. Discov. 1996, 13, 29-47.
36. Schinazi, R. F. Emtricitabine: A viewpoint by Raymond F. Schinazi. Drugs 2003, 63, 2425-2426.
37. Schinazi, R. F.; Lloyd, R. M., Jr.; Nguyen, M. H.; Cannon, D. L.; McMillan, A.; Ilksoy, N.; Chu, C. K.; Liotta, D. C.; Bazmi, H. Z.; Mellors, J. W. Characterization of human immunodeficiency viruses resistant to oxathiolane-cytosine nucleosides. Antimicrob. Agents Chemother. 1993, 37, 875-881. https://doi.org/10.1128/aac.37.4.875.
38. Schinazi, R. F.; McMillan, A.; Cannon, D.; Mathis, R.; Lloyd, R. M.; Peck, A.; Sommadossi, J. P.; St Clair, M.; Wilson, J.; Furman, P. A.; et al. Selective inhibition of human immunodeficiency viruses by racemates and enantiomers of cis-5-fluoro-1-[2-(hydroxymethyl)-1,3-oxathiolan-5-yl] cytosine. Antimicrob. Agents Chemother. 1992, 36, 2423-2431. https://doi.org/10.1128/aac.36.11.2423.
39. Ray, A. S.; Murakami, E.; Peterson, C. N.; Shi, J.; Schinazi, R. F.; Anderson, K. S. Interactions of enantiomers of 2′,3′-didehydro-2′,3′-dideoxy-fluorocytidine with wild type and m184v mutant HIV-1 reverse transcriptase. Antivir. Res. 2002, 56, 189-205. https://doi.org/10.1016/s0166-3542 (02) 00102-x.
40. Tisdale, M.; Kemp, S. D.; Parry, N. R.; Larder, B. A. Rapid in vitro selection of human immunodeficiency virus type 1 resistant to 3′-thiacytidine inhibitors due to a mutation in the ymdd region of reverse transcriptase. Proc. Natl. Acad. Sci. USA 1993, 90, 5653-5656. https://doi.org/10.1073/pnas.90.12.5653.
41. HIV Drug Resistance Database. Available online: https://hivdb.stanford.edu/dr-summary/resistance-notes/NRTI/(accessed on 14 Jul. 2022).
42. Feng, J. Y.; Myrick, F. T.; Margot, N. A.; Mulamba, G. B.; Rimsky, L.; Borroto-Esoda, K.; Selmi, B.; Canard, B. Virologic and enzymatic studies revealing the mechanism of K65R- and Q151M-associated HIV-1 drug resistance towards emtricitabine and lamivudine. Nucleosides Nucleotides Nucleic Acids 2006, 25, 89-107. https://doi.org/10.1080/15257770500379157.
43. Seitz, S.; Habjanic, J.; Schutz, A. K.; Bartenschlager, R. The hepatitis B virus envelope proteins: molecular gymnastics throughout the viral life cycle. Annu. Rev. Virol. 2020, 7, 263-288. https://doi.org/10.1146/annurev-virology-092818-015508.
44. Amblard, F.; Boucle, S.; Bassit, L.; Chen, Z.; Sari, O.; Cox, B.; Verma, K.; Ozturk, T.; Ollinger-Russell, O.; Schinazi, R. F. Discovery and structure activity relationship of glyoxamide derivatives as anti-hepatitis B virus agents. Bioorganic Med. Chem. 2021, 31, 115952. https://doi.org/10.1016/j.bmc.2020.115952.
45. Amblard, F.; Boucle, S.; Bassit, L.; Cox, B.; Sari, O.; Tao, S.; Chen, Z.; Ozturk, T.; Verma, K.; Russell, O.; et al. Novel hepatitis B virus capsid assembly modulator induces potent antiviral responses in vitro and in humanized mice. Antimicrob. Agents Chemother. 2020, 64, e01701-19. https://doi.org/10.1128/AAC.01701-19.
46. Hurwitz, S.; McBrearty, N.; Arzumanyan, A.; Bichenkov, E.; Tao, S.; Bassit, L.; Chen, Z.; Kohler, J. J.; Amblard, F.; Feitelson, M. A.; et al. Studies on the efficacy, potential cardiotoxicity and monkey pharmacokinetics of GLP-26 as a potent hepatitis B virus capsid assembly modulator. Viruses 2021, 13, 114. https://doi.org/10.3390/v13010114.
47. Verbinnen, T.; Tan, Y.; Wang, G.; Dehertogh, P.; Vergauwen, K.; Neefs, J. M.; Jacoby, E.; Lenz, O.; Berke, J. M. Anti-HBV activity of the HBV capsid assembly modulator jnj-56136379 across full-length genotype a-h clinical isolates and core site-directed mutants in vitro. J. Antimicrob. Chemother. 2020, 75, 2526-2534. https://doi.org/10.1093/jac/dkaa179.
48. Ruan, L.; Hadden, J. A.; Zlotnick, A. Assembly properties of hepatitis B virus core protein mutants correlate with their resistance to assembly-directed antivirals. J. Virol. 2018, 92, e01018-e01082.
49. Yan, Z.; Wu, D.; Hu, H.; Zeng, J.; Yu, X.; Xu, Z.; Zhou, Z.; Zhou, X.; Yang, G.; Young, J. A. T.; et al. Direct inhibition of hepatitis B e antigen by core protein allosteric modulator. Hepatology 2019, 70, 11-24. https://doi.org/10.1002/hep.30514.
50. Zhou, T.; Guo, H.; Guo, J. T.; Cuconati, A.; Mehta, A.; Block, T. M. Hepatitis b virus e antigen production is dependent upon covalently closed circular (ccc) DNA in hepad38 cell cultures and may serve as a cccdna surrogate in antiviral screening assays. Antivir. Res. 2006, 72, 116-124. https://doi.org/10.1016/j.antiviral.2006.05.006.
51. Patel, D.; Patel, J. S.; Ytreberg, F. M. Implementing and assessing an alchemical method for calculating protein-protein binding free energy. J. Chem. Theory Comput. 2021, 17, 2457-2464. https://doi.org/10.1021/acs.jctc.0c01045.
52. Gapsys, V.; Michielssens, S.; Seeliger, D.; de Groot, B. L. Pmx: Automated protein structure and topology generation for alchemical perturbations. J. Comput. Chem. 2015, 36, 348-354. https://doi.org/10.1002/jcc.23804.
53. Jacobson, M. P.; Friesner, R. A.; Xiang, Z.; Honig, B. On the role of the crystal environment in determining protein side-chain conformations. J. Mol. Biol. 2002, 320, 597-608. https://doi.org/10.1016/s0022-2836 (02) 00470-9.
54. Jacobson, M. P.; Pincus, D. L.; Rapp, C. S.; Day, T. J.; Honig, B.; Shaw, D. E.; Friesner, R. A. A hierarchical approach to all-atom protein loop prediction. Proteins Struct. Funct. Bioinform. 2004, 55, 351-367.
55. Shelley, J. C.; Cholleti, A.; Frye, L. L.; Greenwood, J. R.; Timlin, M. R.; Uchimaya, M. Epik: A software program for pkaprediction and protonation state generation for drug-like molecules. J. Comput.-Aided Mol. Des. 2007, 21, 681-691. https: //doi.org/10.1007/s10822-007-9133-z.
56. Ren, Q.; Liu, X.; Luo, Z.; Li, J.; Wang, C.; Goldmann, S.; Zhang, J.; Zhang, Y. Discovery of hepatitis B virus capsid assembly inhibitors leading to a heteroaryldihydropyrimidine based clinical candidate (gls4). Bioorganic Med. Chem. 2017, 25, 1042-1056. https://doi.org/10.1016/j.bmc.2016.12.017.
57. Günther, S.; Li, B. C.; Miska, S.; Krüger, D. H.; Meisel, H.; Will, H. A novel method for efficient amplification of whole hepatitis B virus genomes permits rapid functional analysis and reveals deletion mutants in immunosuppressed patients. J. Virol. 1995, 69, 5437-5444. https://doi.org/10.1128/JVI.69.9.5437-5444.1995.
58. Sambrook, J.; Fritsch, E. F.; Maniatis, T. (Eds.) Molecular Cloning: A Laboratory Manual; NY. 16.56-16.67; Cold Spring Harbor Laboratory Press: Cold Spring Harbor, NY, USA, 1989.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A predictive model for determining what mutations a virus is likely to develop in response to exposure to a drug that binds to a viral enzyme, comprising:
a) obtaining a crystal structure of the viral enzyme, or a portion thereof, complexed with an inhibitor of the enzyme, where the complex occurs in a binding pocket of the enzyme or portion thereof;
b) creating an in silico model of the crystal structure of the viral enzyme complexed with the inhibitor, which model maps out the amino acid sequence of the enzyme, or portion thereof, c) making changes to the amino acids in the binding pocket, and measuring, in silico, changes in the Gibbs free energy between the viral enzyme, or portion thereof, and the inhibitor, to identify mutations that would tend to cause the inhibitor to no longer remain bound to the binding pocket,
d) obtaining a crystal structure of one or more substrates to which the enzyme binds when exerting its enzymatic function,
e) creating an in silico model of the crystal structure of the viral enzyme complexed with the one or more substrates, which model maps out the amino acid sequence of the enzyme, or portion thereof, and
f) making changes to the amino acids in the binding pocket mirroring the changes identified in step c), and
g) identifying those changes to the amino acids in the binding pocket that do not result in a likelihood that the enzyme will not remain bound to the binding pocket,
wherein where a mutation would likely cause the inhibitor to no longer bind with high affinity to the enzyme, or portion thereof, but not significantly decrease the affinity of the enzyme to the one or more substrates, this correlates to a mutation that the virus would likely make upon exposure to the inhibitor.

2. The predictive model of claim 1, wherein the enzyme is the SARS-COV-23CLpro.

3. The predictive model of claim 2, wherein the inhibitor is PF-07321332.

4. The predictive model of claim 1, wherein the enzyme is an HIV or HBV reverse transcriptase.

5. The predictive model of claim 4, wherein the reverse transcriptase inhibitor is Zidovudine, Didanosine, Zalcitabine, Stavudine, Lamivudine, Abacavir, Emtricitabine, Entecavir, or Azvudine.

6. The predictive model of claim 1 further comprising repeating steps a) through g) with a second inhibitor, to determine whether there are mutations predicted with respect to the first inhibitor that are not predicted with respect to the second inhibitor, wherein if there are mutations predicted to be associated with the first inhibitor, which do not significantly reduce the affinity between the enzyme and the one or more substrates, that are not associated with the second inhibitor, it is predicted that the second inhibitor will remain effective against the enzyme if the virus mutates in response to the first inhibitor.

7. The predictive model of claim 4, wherein the enzyme is wherein the enzyme is the SARS-COV-23CLpro.

8. The predictive model of claim 7, wherein the first inhibitor is PF-07321332.

9. A method of treating a viral infection, comprising:
a) screening a biological sample obtained from a patient infected with a virus to identify the presence of one or more mutations in the viral enzyme,
b) comparing the mutations with the mutations identified using the method of claim 1, wherein if one or more mutations identified using the predictive model of claim 1 are identified, this is indicative that the inhibitor will not be successful in treating the viral infection, and if none of the predicted mutations are identified, then the inhibitor will likely be successful in treating the viral infection.

10. The method of claim 9, further comprising, if the viral enzyme is not likely to be successfully inhibited using the inhibitor, repeating the predictive model with a second inhibitor, to determine whether the second inhibitor would likely be successful in treating the viral infection.

11. The method of claim 10, wherein if the second inhibitor would likely be successful in treating the viral infection, the patient is treated with the second inhibitor.

12. The method of claim 9, wherein if the second inhibitor would not likely be successful in treating the viral infection, the patient is treated with a treatment regimen that does not include the first or second inhibitor.
